(19) United States
(12) Patent Application Publication
(10) Pub. No.: US 2004/0022796 A1
(43) Pub. Date: Feb. 5, 2004

(54) Using heat shock proteins and alpha-2-macroglobulins to increase the immune response to vaccines comprising heat shock protein-peptide complexes or alpha-2-macroglobulin-peptide complexes

(75) Inventor: Pramod K. Srivastava, Avon, CT (US)

Correspondence Address: Pennie & Edmonds LLP, 1155 Avenue of the Americas, New York, NY, 10036-2711, US

(73) Assignee: University of Connecticut Health Center

(21) Appl. No.: 10/427,857

(22) Filed: May 1, 2003

Related U.S. Application Data
(60) Provisional application No. 60/377,484, filed on May 2, 2002.

(51) Int. Cl. A61K 39/00

(52) U.S. Cl. 424/185.1

(57) ABSTRACT

The present invention provides a method of improving or prolonging a subject's immune response to a vaccine composition comprising heat shock protein (HSP)-peptide complexes or alpha-2-macroglobulin (α2M)-peptide complexes (hereinafter “HSP/α2M vaccine composition”). The HSP-peptide complexes or α2M-peptide complexes of the vaccine composition comprise HSP(s) or α2M complexed to a component against which an immune response is desired to be induced. In particular the invention is directed to methods of improving or prolonging a subject's immune response comprising administering an HSP/α2M vaccine composition in conjunction with a preparation comprising HSP or α2M, alone or complexed to a peptide that is not the component against which an immune response is desired to be induced (hereinafter “HSP/α2M preparation”), i.e., the HSP/α2M preparation does not display the immunogenicity of the component. In particular, HSP/α2M vaccine compositions are administered in conjunction with HSP/α2M preparation to improve or prolong the immune response of a subject against an infectious disease or cancer.

This application claims the benefit of U.S. Provisional Application No. 60/377,484, filed May 2, 2002, which is incorporated by reference herein in its entirety.

### 1. INTRODUCTION

The present invention provides a method of improving or prolonging a subject's immune response to a vaccine composition comprising heat shock protein (HSP)-peptide complexes or alpha-2-macroglobulin (α2M)-peptide complexes (hereinafter “HSP/α2M vaccine composition”). The HSP-peptide complexes or α2M-peptide complexes of the vaccine composition comprise HSP(s) or α2M complexed to a component against which an immune response is desired to be induced. The invention is directed to methods of improving or prolonging a subject's immune response comprising administering an HSP/α2M vaccine composition in conjunction with a preparation comprising HSP or α2M, alone or complexed to a peptide that is not the component against which an immune response is desired to be induced (hereinafter “HSP/α2M preparation”). In particular, HSP/α2M vaccine compositions are administered in conjunction with HSP/α2M preparation to improve or prolong the immune response of a subject against an infectious disease or cancer.

### 2. BACKGROUND OF THE INVENTION

Citation or discussion of a reference herein shall not be construed as an admission that such is prior art to the present invention.

2.1. Vaccines

Vaccination has eradicated certain diseases such as polio, tetanus, chicken pox, and measles in many countries. This approach has exploited the ability of the immune system to resist and prevent infectious diseases.

Traditional ways of preparing vaccines include the use of inactivated or attenuated pathogens. A suitable inactivation of the pathogenic microorganism renders it harmless as a biological agent but does not destroy its immunogenicity. Injection of these “killed” particles into a host will then elicit an immune response capable of preventing a future infection with a live microorganism. However, a major concern in the use of inactivated pathogens as vaccines is the failure to inactivate all the microorganisms. Even when this is accomplished, since killed pathogens do not multiply in their host, or for other unknown reasons, the immunity achieved is often incomplete, short lived and requires multiple immunizations. Finally, the inactivation process may alter the microorganism's antigens, rendering them less effective as immunogens.

Attenuation refers to the production of strains of pathogenic microorganisms which have essentially lost their disease-producing ability. One way to accomplish this is to subject the microorganism to unusual growth conditions and/or frequent passage in cell culture. Mutants are then selected which have lost virulence but yet are capable of eliciting an immune response. Attenuated pathogens often make good immunogens as they actually replicate in the host cell and elicit long lasting immunity. However, several problems are encountered with the use of live vaccines, the most worrisome being insufficient attenuation and the risk of reversion to virulence.

An alternative to the above methods is the use of subunit vaccines. This involves immunization only with those components which contain the relevant immunological material. A new promising alternative is the use of DNA or RNA as vaccines. Such genetic vaccines have progressed from an idea to entities being studied in clinical trials (See, Weiner and Kennedy, July 1999, Scientific American, pp. 50-57).

Vaccines are often formulated and inoculated with various adjuvants. The adjuvants aid in attaining a more durable and higher level of immunity using small amounts of antigen or fewer doses than if the immunogen were administered alone. The mechanism of adjuvant action is unpredictable, complex and not completely understood (See Suzue, et al., 1996, Basel: Birkhauser Verlag, 454-55).

Because of the risks associated with inactivated and attenuated pathogens, the ability to boost or amplify an immune response to minimal quantities of a vaccine would be ideal and advantageous. Furthermore, as the mechanism of adjuvants is not completely understood and is still unpredictable, alternative methods of boosting a subject's immune response with current methods of vaccination is highly desirable.

2.2. Immune Responses

An organism's immune system reacts with two types of responses to pathogens or other harmful agents—humoral response and cell-mediated response (See Alberts, B. et al., 1994, Molecular Biology of the Cell. 1195-96). When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells—cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells, for example (See Alberts, B. et al., 1994, Molecular Biology of the Cell. 1228). Both cytotoxic T cells and helper T cells recognize antigen in the form of peptide fragments that are generated by the degradation of foreign protein antigens inside the target cell, and both, therefore, depend on major histocompatibility complex (MHC) molecules, which bind these peptide fragments, carry them to the cell surface, and present them there to the T cells (See Alberts, B. et al., 1994, Molecular Biology of the Cell. 1228). MHC molecules are typically found in abundance on antigen-presenting cells (APCs).

2.3. Heat Shock Proteins

Heat shock proteins (HSPs), which are also referred to interchangeably herein as stress proteins, can be selected from among any cellular protein that satisfies the following criteria. It is a protein whose intracellular concentration increases when a cell is exposed to a stressful stimuli, it is capable of binding other proteins or peptides, and it is capable of releasing the bound proteins or peptides in the presence of adenosine triphosphate (ATP) or low pH. In addition, it has been discovered that the Hsp-60, Hsp-70 and Hsp-90 families are composed of proteins related to the stress proteins in amino acid sequence, for example, having greater than 35% amino acid identity, but whose expression levels are not altered by stressful stimuli. Accordingly, it is contemplated that the definition of stress protein, as used herein, embraces other proteins, muteins, analogs, and variants thereof having at least 35% to 55%, preferably 55% to 75%, and most preferably 75% to 85% amino acid identity with members of the three families whose expression levels in a cell are stimulated in response to stressful stimuli.

The first stress proteins to be identified were the HSPs. As their name implies, HSPs are synthesized by a cell in response to heat shock. To date, three major families of HSPs have been identified based on molecular weight. The families have been called hsp60, hsp70, and hsp90 where the numbers reflect the approximate molecular weight of the stress proteins in kilodaltons. Many members of these families were found subsequently to be induced in response to other stressful stimuli including, but not limited to, nutrient deprivation, metabolic disruption, oxygen radicals, and infection with intracellular pathogens. (See Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677), the disclosures of which are incorporated herein by reference. It is contemplated that hsps/stress proteins belonging to all of these three families can be used in the practice of the instant invention.

HSPs are intracellular molecules that are abundant, soluble, and highly conserved. As intracellular chaperones, HSPs participate in many biochemical pathways of protein maturation and function active during times of stress and normal cellular homeostasis. Many stresses can disrupt the three-dimensional structure, or folding, of a cell's proteins. Left uncorrected, mis-folded proteins form aggregates that may eventually kill the cell. HSPs bind to those damaged proteins, helping them refold into their proper conformations. In normal (unstressed) cellular homeostasis, HSPs are required for cellular metabolism. HSPs help newly synthesized polypeptides fold and thus prevent premature interactions with other proteins. Also, HSPs aid in the transport of proteins throughout the cell's various compartments.

The major HSPs can accumulate to very high levels in stressed cells, but they occur at low to moderate levels in cells that have not been stressed. For example, the highly inducible mammalian hsp70 is hardly detectable at normal temperatures but becomes one of the most actively synthesized proteins in the cell upon heat shock (Welch et al., 1985, J. Cell. Biol. 101:1198-1211). In contrast, hsp90 and hsp60 proteins are abundant at normal temperatures in most, but not all, mammalian cells and are further induced by heat (Lai et al., 1984, Mol. Cell. Biol. 4:2802-2810; van Bergen en Henegouwen et al., 1987, Genes Dev. 1:525-531).

HSPs have been found to have immunological and antigenic properties. Immunization of mice with gp96 or p84/86 isolated from a particular tumor rendered the mice immune to that particular tumor, but not to antigenically distinct tumors (Srivastava, P. K. et al., 1988, Immunogenetics 28:205-207; Srivastava, P. K. et al., 1991, Curr. Top. Microbiol. Immunol. 167:109-123). Further, hsp70 was shown to elicit immunity to the tumor from which it was isolated but not to antigenically distinct tumors. However, hsp70 depleted of peptides was found to lose its specific immunogenic activity (Udono, M., and Srivastava, P. K., 1993, J. Exp. Med. 178:1391-1396). These observations suggested that the heat shock proteins are not antigenic per se, but form noncovalent complexes with antigenic peptides, and the complexes can elicit specific immunity to the antigenic peptides (Srivastava, P. K., 1993, Adv. Cancer Res. 62:153-177; Udono, H. et al., 1994, J. Immunol., 152:5398-5403; Suto, R. et al., 1995, Science, 269:1585-1588). Recently, hsp60 and hsp70 have been found to stimulate production of proinflammatory cytokines, such as TNFα and IL-6, by monocytes, macrophages, or cytotoxtic T cells (Breloer et al., 1999, J. Immunol. 162:3141-3147; Chen et al., 1999, J. Immunol. 162:3212-3219; Ohashi et al., 2000, J. Immunol. 164:558-561; Asea et al., 2000, Nature Medicine, 6:435-442; Todryk et al., 1999, J. Immunol. 163:1398-1408). Hsp70 has also been shown to target immature dendritic cells and make them more able to capture antigens (Todryk et al., J. Immunol. 163:1398-1408). It has been postulated that release of or induction of expression of hsp60 and hsp70, e.g., due to cell death, may serve to signal that an immune reaction should be raised (Chen et al., 1999, J. Immunol. 162:3212-3219; Ohashi et al., 2000, J. Immunol. 164:558-561; Todryk et al., 1999, J. Immunol. 163:1398-1408; Basu et al. Intl. Immunol. 2000 vol 12: 1539-1546).

The use of noncovalent complexes of HSP and peptide, purified from cancer cells, for the treatment and prevention of cancer has been described in U.S. Pat. Nos. 5,750,119, 5,837,251, and 6,017,540.

The use of HSP-peptide complexes for sensitizing antigen presenting cells in vitro for use in adoptive immunotherapy is described in U.S. Pat. Nos. 5,985,270 and 5,830,464.

HSP-peptide complexes can also be isolated from pathogen-infected cells and used for the treatment and prevention of infection caused by the pathogen, such as viruses, and other intracellular pathogens, including bacteria, protozoa, fungi and parasites; see U.S. Pat. Nos. 5,961,979, and 6,048,530.

Immunogenic HSP-peptide complexes can also be prepared by in vitro complexing of HSPs and antigenic peptides, and the uses of such complexes for the treatment and prevention of cancer and infectious diseases has been described in U.S. Pat. Nos. 5,935,576, and 6,030,618. The use of heat shock protein in combination with a defined antigen for the treatment of cancer and infectious diseases have also been described in PCT publication WO97/06821 dated Feb. 27, 1997.

The purification of HSP-peptide complexes from cell lysate has been described previously; see for example, U.S. Pat. Nos. 5,750,119, and 5,997,873.

2.4. Alpha-2-Macroglobulin

The alpha-2-macroglobulins are members of a protein superfamily of structurally related proteins which also comprises complement components C3, C4 and C5. The human plasma protein alpha-2-macroglobulin (α2M) is a 720 kDa homotetrameric protein primarily known as proteinase inhibitor and plasma and inflammatory fluid proteinase scavenger molecule (for review see Chu and Pizzo, 1994, Lab. Invest. 71:792). Alpha-2-macroglobulin is synthesized as a 1474 amino acid precursor, the first 23 of which function as a signal sequence that is cleaved to yield a 1451 amino acid mature protein (Kan et al., 1985, Proc. Natl. Acad. Sci. U.S.A. 82:2282-2286).

α2M promiscuously binds to proteins and peptides with nucleophilic amino acid side chains in a covalent manner (Chu et al., 1994, Ann. N.Y. Acad. Sci. 737:291-307) and targets them to cells which express the α2M receptor (α2MR) (Chu and Pizzo, 1993, J. Immunol. 150:48). Binding of α2M to the α2MR is mediated by the C-terminal portion of α2M (Holtet et al., 1994, FEBS Lett. 344:242-246) and key residues have been identified (Nielsen et al., 1996, J. Biol. Chem. 271:12909-12912).

Generally known for inhibiting protease activity, α2M binds to a variety of proteases thorough multiple binding sites (see, e.g., Hall et al., 1981, Biochem. Biophys. Res. Commun.100(1):8-16). Protease interaction with α2M results in a complex structural rearrangement called transformation, which is the result of a cleavage within the “bait” region of α2M after the proteinase becomes “trapped” by thioesters. The conformational change exposes residues required for receptor binding, allowing the α2M-proteinase complex to bind to the α2MR. Methylamine can induce similar conformational changes and cleavage as that induced by proteinases. The uncleaved form of α2M, which is not recognized by the receptor, is often referred to as the “slow” form (s-α2M). The cleaved form is referred to as the “fast” form (f-α2M) (reviewed by Chu et al., 1994, Ann. N.Y. Acad. Sci. 737:291-307).

Studies have shown that, in addition to its proteinase-inhibitory functions, α2M, when complexed to antigens, can enhance the antigens' ability to be taken up by antigen presenting cells such as macrophages and presented to T cell hybridomas in vitro by up to two orders of magnitude (Chu and Pizzo, 1994, Lab. Invest. 71:792), and induce T cell proliferation (Osada et al., 1987, Biochem. Biophys. Res. Commun.146:26-31). Further evidence suggests that complexing antigen with α2M enhances antibody production by crude spleen cells in vitro (Osada et al., 1988, Biochem. Biophys. Res. Commun. 150:883) and elicits an in vivo antibody responses in experimental rabbits (Chu et al., 1994, J. Immunol. 152:1538-1545) and mice (Mitsuda et al., 1993, Biochem. Biophys. Res. Commun. 101:1326-1331). However, none of these studies have shown whether α2M-antigen complexes are capable of eliciting cytotoxic T cell responses in vivo.

α2M can form complexes with antigens, which are taken up by antigen presenting cells (“APCs”) via the α2MR, also known as LDL (low-density lipoprotein) Receptor-Related Protein (“LRP”) or CD91 (see PCT/US01/18047, which is incorporated by reference herein in its entirety). α2M directly competes for the binding of heat shock protein gp96 to the α2MR, indicating that α2M and HSPs may bind to a common recognition site on the α2MR (Binder et al, 2000, Nature Immunology 1(2), 151-154). Additionally, α2M-antigenic peptide complexes prepared in vitro can be administered to animals to generate a cytotoxic T cell response specific to the antigenic molecules (Binder et al., 2001, J. Immunol. 166:4968-72). Thus, because HSPs and α2M have a number of common functional attributes, such as the ability to bind peptide, the recognition and uptake by the α2MR, and the stimulation of a cytotoxic T cell response, α2M can be used for immunotherapy against cancer and infectious disease.

### 3. SUMMARY OF THE INVENTION

The present invention provides a method of improving or prolonging a subject's immune response to a vaccine composition comprising heat shock protein (HSP)-peptide complexes or alpha-2-macroglobulin (α2M)-peptide complexes (hereinafter “HSP/α2-M vaccine composition”). The HSP-peptide complexes or α2M-peptide complexes of the vaccine composition comprise HSP(s) or α2M complexed to a component against which an immune response is desired to be induced. The invention is directed to methods of improving or prolonging a subject's immune response comprising administering an HSP/α2M vaccine composition in conjunction with a preparation comprising HSP or α2M, alone or complexed to a peptide that is not the component against which an immune response is desired to be induced (hereinafter “HSP/α2M preparation”). In particular, HSP/α2M vaccine compositions are administered in conjunction with HSP/α2M preparation to improve or prolong the immune response of a subject against an infectious disease or cancer.

Accordingly, in one embodiment, the invention encompasses a method of improving or prolonging a subject's immune response to an HSP vaccine composition useful for the prevention or treatment of cancer or infectious disease that comprises HSP-peptide complexes comprising HSP complexed to a peptide that displays the antigenicity of an antigen of an agent of an infectious disease or a tumor specific or tumor associated antigen of a type of cancer by administering the HSP vaccine composition in conjunction with an HSP preparation comprising HSP, alone or complexed to a peptide that does not display any antigenicity of an antigen of an agent of an infectious disease or a tumor specific or tumor associated antigen of a type of cancer. In an alternate embodiment, the subject's immune response to an HSP vaccine composition useful for the prevention or treatment of cancer or infectious disease is improved or prolonged by administering the HSP vaccine composition in conjunction with a α2M preparation comprising α2M, alone or complexed to a peptide that does not display the antigenicity of the antigen of an agent of an infectious disease or tumor specific or tumor associated antigen of a type of cancer.

In another embodiment, the invention encompasses a method of improving or prolonging a subject's immune response to a α2M vaccine composition useful for the prevention or treatment of cancer or infectious disease that comprises α2M-peptide complexes comprising α2M complexed to a peptide that displays the antigenicity of an antigen of an agent of an infectious disease or a tumor specific or tumor associated antigen of a type of cancer by administering the α2M vaccine composition in conjunction with a α2M preparation comprising α2M, alone or complexed to a peptide that does not display any antigenicity of an antigen of an agent of an infectious disease or a tumor specific or tumor associated antigen of a type of cancer. In an alternate embodiment, the subject's immune response to a α2M vaccine composition useful for the prevention or treatment of cancer or infectious disease is improved or prolonged by administering the α2M vaccine composition in conjunction with an HSP preparation comprising HSP alone or complexed to a peptide that does not display the antigenicity of the antigen of an agent of an infectious disease or tumor specific or tumor associated antigen of a type of cancer.

In one embodiment of the invention, the method for inducing an immune response comprises administering to the subject an HSP/α2M vaccine composition comprising an HSP or an α2M, complexed to a component against which an immune response is desired to be induced; and administering to the subject an HSP preparation, wherein the immune response against the component is not elicited in the absence of the administering of the HSP/α2M vaccine composition. The HSP preparation does not display the immunogenicity of the component. Preferably, the HSP preparation alone cannot elicit an immune response against the component in the absence of the administering of the HSP/α2M vaccine composition. Preferably, the method can increase the magnitude and duration of the immune response to the component of interest relative to that obtained in the absence of administering to the subject an HSP preparation. In a preferred embodiment, the HSP/α2M vaccine composition and the HSP preparation are not present in admixture.

In another embodiment of the invention, the method for inducing an immune response comprises administering to the subject an HSP/α2M vaccine composition comprising an HSP or α2M complexed to a component against which an immune response is desired to be induced; and administering to the subject an α2M preparation, wherein the immune response against the component is not elicited in the absence of the administering of the HSP/α2M vaccine composition. The α2M preparation does not display the immunogenicity of the component. Preferably, the α2M preparation alone cannot elicit an immune response against the component in the absence of the administering of the HSP/α2M vaccine composition. Preferably, the method can increase the magnitude and duration of the immune response to the component of interest relative to that obtained in the absence of administering to the subject an α2M preparation. In a preferred embodiment, the HSP/α2M vaccine composition and the α2M preparation are not present in admixture.

In another embodiment, the invention provides for a method of inducing an immune response by a sub-immunogenic amount of an HSP/α2M vaccine composition, wherein the HSP preparation facilitates the induction of an immune response by an amount of HSP/α2M vaccine composition which is otherwise insufficient for inducing the immune response when used alone. In particular, the method comprises the steps of (a) administering to the subject an amount of an HSP preparation; and (b) administering to the subject an HSP/α2M vaccine composition comprising an HSP or α2M complexed to a component against which an immune response is desired to be induced in an amount that is sub-immunogenic in the absence of step(a), whereby an immune response to said component is induced in the subject, and wherein the HSP preparation does not display the immunogenicity of the component. The HSP preparation does not elicit an immune response against said component in the absence of said administering of the HSP/α2M vaccine composition. In a preferred embodiment, the HSP/α2M vaccine composition and the HSP preparation are not present in admixture.

In another embodiment, the invention provides for a method of inducing an immune response by a sub-immunogenic amount of an HSP/α2M vaccine composition, wherein the α2M preparation facilitates the induction of an immune response by an amount of HSP/α2M vaccine composition which is otherwise insufficient for inducing the immune response when used alone. In particular, the method comprises the steps of (a) administering to the subject an amount of an α2M preparation; and (b) administering to the subject an HSP/α2M vaccine composition comprising an HSP or α2M complexed to a component against which an immune response is desired to be induced in an amount that is sub-immunogenic in the absence of step(a), whereby an immune response to said component is induced in the subject, and wherein the α2M preparation does not display the immunogenicity of the component. The α2M preparation does not elicit an immune response against said component in the absence of said administering of the HSP/α2M vaccine composition. In a preferred embodiment, the HSP/α2M vaccine composition and the α2M preparation are not present in admixture.

In yet another embodiment, the invention provides a method of treating or preventing an infectious disease in a subject comprising administering to the subject an HSP/α2M vaccine composition comprising an HSP or α2M complexed to a component that displays the antigenicity of an infectious agent that causes the infectious disease; and administering to the subject an amount of a heat shock protein preparation effective in combination with the HSP/α2M vaccine composition to induce or increase an immune response to the component in the subject. The heat shock protein preparation does not display the immunogenicity of the component. In a preferred embodiment, the HSP/α2M vaccine composition and the α2M preparation are not present in admixture.

In yet another embodiment, the invention provides a method of treating or preventing an infectious disease in a subject comprising administering to the subject an HSP/α2M vaccine composition comprising an HSP or α2M complexed to a component that displays the antigenicity of an infectious agent that causes the infectious disease; and administering to the subject an amount of an α2M preparation effective in combination with the HSP/α2M vaccine composition to induce or increase an immune response to the component in the subject. The α2M preparation does not display the immunogenicity of the component. In a preferred embodiment, the HSP/α2M vaccine composition and the α2M preparation are not present in admixture.

In yet another embodiment, the invention provides a method of treating or preventing a cancer in a subject comprising administering to the subject an HSP/α2M vaccine composition comprising an HSP or α2M complexed to a component that displays the antigenicity of a cancer cell; and administering to the subject an amount of a heat shock protein preparation effective to induce or increase an immune response in the subject to the component wherein the heat shock protein preparation does not display the immunogenicity of the component. In a preferred embodiment, the HSP/α2M vaccine composition and the α2M preparation are not present in admixture.

In yet another embodiment, the invention provides a method of treating or preventing a cancer in a subject comprising administering to the subject an HSP/α2M vaccine composition comprising an HSP or α2M complexed to a component that displays the antigenicity of a cancer cell; and administering to the subject an amount of an α2M preparation effective to induce or increase an immune response in the subject to the component wherein the α2M preparation does not display the immunogenicity of the component. In a preferred embodiment, the HSP/α2M vaccine composition and the α2M preparation are not present in admixture.

In yet another embodiment, the invention provides a method of inducing an immune response by an HSP/α2M vaccine composition in a subject comprising administering to the subject a heat shock protein preparation; and administering to the subject an HSP/α2M vaccine composition comprising an HSP or α2M complexed to a component against which an immune response is desired to be induced, the HSP/α2M vaccine composition being in an amount that is sub-immunogenic for the component in the absence of the heat shock protein preparation. The heat shock protein preparation does not display the immunogenicity of the component. In a preferred embodiment, the HSP/α2M vaccine composition and the heat shock protein preparation are not present in admixture.

In yet another embodiment, the invention provides a method of inducing an immune response by an HSP/α2M vaccine composition in a subject comprising administering to the subject an α2M preparation; and administering to the subject an HSP/α2M vaccine composition comprising an HSP or α2M complexed to a component against which an immune response is desired to be induced, the HSP/α2M vaccine composition being in an amount that is sub-immunogenic for the component in the absence of the HSP/α2M vaccine composition. The α2M preparation does not display the immunogenicity of the component. In a preferred embodiment, the HSP/α2M vaccine composition and the α2M preparation are not present in admixture.

In these above-mentioned embodiments of the invention, the HSP preparation and the α2M preparation do not elicit an immune response against the component in the absence of the administration of the HSP/α2M vaccine composition. Preferably, the HSP preparation and the α2M preparation do not display the immunogenicity of the component in the HSP/α2M vaccine composition. The immunogenicity of a heat shock protein preparation or an α2M preparation can be tested in vivo or in vitro by any method known in the art, such as but not limited to those described in section 5.6.

In various embodiments, the HSP preparation or α2M preparation is administered into a subject before the administration of an HSP/α2M vaccine composition. Alternatively, the HSP preparation or α2M preparation is administered to the subject concurrently with the administration of an HSP/α2M vaccine composition, but not in admisture. The HSP preparation or α2M preparation can also be administered to the subject after the administration of an HSP/α2M vaccine composition. Preferably, the subject is mammalian, or, more specifically, human.

Without being bound by any theory, HSPs induce secretion of cytokines and surface expression of antigen-presenting and co-stimulatory molecules, both of which are important for the priming and maintenance of T cell responses. It is also believed that α2M induces secretion of cytokines and surface expression of antigen-presenting and co-stimulatory molecules. Applicant's experimentation with CD11b+ cell activation shows that the presence of HSPs in the extracellular milieu induces interleukin-1β secretion and surface expression of MHC class II molecules. Furthermore, HSP-peptide complexes and α2M-peptide complexes such as those in an HSP/α2M vaccine composition are taken up by antigen presenting cells, which should lead to the activation of a specific T cell response. Accordingly, it is believed that the HSP preparation or the α2M preparation administered to a subject can boost the effectiveness of the HSP/α2M vaccine composition by prolonging the activation state of T cells.

The HSP preparation used in the methods of the invention is preferably free HSP not bound to any molecule or an HSP-peptide complex that comprises an HSP covalently or noncovalently attached to a peptide that is preferably not the same component against which the immune response is desired.

The α2M preparation used in the methods of the invention is preferably free α2M not bound to any molecule or an α2M-peptide complex that comprises an α2M covalently or noncovalently attached to a peptide that is preferably not the same component against which the immune response is desired.

Also encompassed in the invention are kits comprising one or more containers each containing an HSP preparation in an amount effective to increase an immune response elicited by an HSP/α2M vaccine composition against a component of the HSP/α2M vaccine composition against which an immune response is desired; and one or more containers each containing the HSP/α2M vaccine composition in an amount that, when administered before, concurrently with, or after the administration of the heat shock protein preparation of (a), is effective to induce an immune response against the component. In a preferred embodiment, the HSP/α2M vaccine composition and the heat shock protein preparation are not present in admixture. The invention also encompasses kits comprising one or more containers each containing an α2M preparation in an amount effective to increase an immune response elicited by an HSP/α2M vaccine composition against a component of the HSP/α2M vaccine composition against which an immune response is desired; and one or more containers each containing the HSP/α2M vaccine composition in an amount that, when administered before, concurrently with, or after the administration of the α2M preparation of (a), is effective to induce an immune response against the component. In a preferred embodiment, the HSP/α2M vaccine composition and the α2M preparation are not present in admixture.

### 4. DETAILED DESCRIPTION OF THE INVENTION

The ability to increase or prolong an immune response using the claimed methods with these vaccines is desirable and advantageous. The methods of the invention can also aid the induction of an immune response by an amount of HSP/α2M vaccine composition that is insufficient to induce an immune response if used alone. In a preferred embodiment, the HSP/α2M vaccine composition is an HSP-peptide complex vaccine. In another preferred embodiment, the HSP/α2M vaccine composition is an α2M-peptide complex vaccine. The HSP/α2M vaccine composition may comprise an adjuvant. The HSP/α2M vaccine composition may be administered with one or more adjuvants. The source of the HSP or α2M is preferably an eukaryote, and most preferably a mammal. The subject receiving the treatment is preferably a mammal including, but not limited to, domestic animals, such as cats, dogs; wild animals, including foxes and racoons; livestock and fowl, including horses, cattle, sheep, turkeys and chickens, as well as any rodents. Most preferably, the subject is human. For the purposes of this invention, the HSP preparation is preferably purified, and can include free HSP not bound to any molecule, and molecular complexes of HSP with another molecule, such as a peptide. Accordingly, an HSP preparation may comprise an HSP covalently or noncovalently attached to a peptide. The methods of the invention may or may not require covalent or noncovalent attachment of an HSP to any specific antigens or antigenic peptides prior to administration to a subject. In preferred embodiments, the peptide of the HSP preparation is unrelated to the infectious disease or disorder or particular cancer being treated. An HSP preparation may include crude cell lysate comprising HSP, the amount of lysate corresponding to between 100 to 10⁸ cell equivalents. HSPs can be conveniently purified from most cellular sources as a population of complexes of different peptides non-covalently bound to HSPs. The HSPs can be separated from the non-covalently bound peptides by exposure to low pH and/or adenosine triphosphate, or other methods known in the art.

In various embodiments of the invention, the HSP preparation may include but is not limited to, hsp70, hsp90, gp96, hsp110, grp170 or calreticulin, singly or in combination with each other.

For the purposes of this invention, the α2M preparation is preferably purified, and can include free α2M not bound to any molecule, and molecular complexes of α2M with another molecule, such as a peptide. Accordingly, an α2M preparation may comprise an α2M covalently or noncovalently attached to a peptide. The methods of the invention may or may not require covalent or noncovalent attachment of an α2M to any specific antigens or antigenic peptides prior to administration to a subject. In preferred embodiments, the peptide of the α2M preparation is unrelated to the infectious disease or disorder or particular cancer being treated. An α2M preparation may include crude cell lysate comprising α2M, the amount of lysate corresponding to between 100 to 10⁸ cell equivalents. α2M can be conveniently purified from most cellular sources as a population of complexes of different peptides non-covalently bound to α2Ms. The α2M can be separated from the non-covalently bound peptides by exposure to low pH and/or adenosine triphosphate, or other methods known in the art.

In preferred embodiments, the HSP-peptide vaccine composition comprises complexes of HSP and a peptide which displays the antigenicity of an antigen of the agent of infectious disease or of a tumor specific antigen or tumor associated antigen of the type of cancer being treated. More preferably, for the treatment of infectious disease, the HSP preparation comprises noncovalent HSP-peptide complexes isolated from a cell infected with an infectious agent (or an infectious variant thereof displaying the antigenicity thereof) that causes the infectious disease. More preferably, for treatment of a type of cancer, the HSP-peptide vaccine composition comprises noncovalent HSP-peptide complexes isolated from cancerous tissue of said type of cancer or a metastasis thereof, which can be from the patient (autologous) or not (allogeneic).

In various embodiments of the invention, the HSP-peptide vaccine composition may include but is not limited to, hsp70, hsp90, gp96, hsp110, grp170 or calreticulin, singly or in combination with each other.

In preferred embodiments, the α2M-peptide vaccine composition comprises complexes of α2M and a peptide which displays the antigenicity of an antigen of the agent of infectious disease or of a tumor specific antigen or tumor associated antigen of the type of cancer being treated. For the treatment of infectious disease, the α2M-peptide vaccine composition can comprise noncovalent α2M-peptide complexes isolated from a cell infected with an infectious agent (or an infectious variant thereof displaying the antigenicity thereof) that causes the infectious disease. For treatment of a type of cancer, the α2M preparation comprises noncovalent α2M-peptide complexes isolated from cancerous tissue of said type of cancer or a metastasis thereof, which can be from the patient (autologous) or not (allogeneic).

In various embodiments, the source of the HSP and the α2M is preferably an eukaryote, more preferably a mammal, and most preferably a human. Accordingly, the HSP preparation used by the methods of the invention includes eukaryotic HSPs, mammalian HSPs and human HSPs. The α2M preparation includes eukaryotic α2M, mammalian α2M and human α2M. The eukaryotic source from which the HSP preparation or α2M preparation is derived and the subject receiving the HSP preparation or the α2M preparation, respectively, are preferably the same species.

This invention encompasses methods of treatment that provide better therapeutic profiles than the administration of the HSP/α2M vaccine composition alone. In another embodiment, the invention encompasses methods of treatment that provide better therapeutic profiles than the administration of the HSP vaccine composition alone. In another embodiment, the invention encompasses methods of treatment that provide better therapeutic profiles than the administration of the α2M vaccine composition alone. Encompassed by the invention are methods wherein the administration of a treatment modality with an HSP preparation or an α2M preparation has additive potency or additive therapeutic effect. The invention also encompasses synergistic outcomes where the therapeutic efficacy is greater than additive. Preferably, such administration of an HSP/α2M vaccine composition with an HSP preparation or with an α2M preparation also reduces or avoids unwanted or adverse effects. Given the invention, in certain embodiments, doses of HSP/α2M vaccine composition can be reduced or administered less frequently, preferably increasing patient compliance, improving therapy and/or reducing unwanted or adverse effects. In a specific embodiment, lower or less frequent doses of HSP/α2M vaccine composition are administered to reduce or avoid unwanted effects. Alternatively, doses of HSP preparation and doses of α2M preparation can be reduced or administered less frequently if administered with an HSP/α2M vaccine composition. Preferably, the HSP preparation of α2M preparation administered to a subject can boost the effectiveness of the HSP/α2M vaccine composition by prolonging the activation state of T cells.

Generally, the HSP preparation or α2M preparation is separately administered from the HSP/α2M vaccine composition. In a preferred embodiment, if the component of the HSP/α2M vaccine composition is a peptide complexed to a heat shock protein, the HSP vaccine composition and the heat shock protein preparation are not present in admixture. In another preferred embodiment, if the component of the vaccine composition is a peptide complexed to an α2M, the α2M vaccine composition and the α2M preparation are not present in admixture.

The HSP preparation or the α2M preparation can be administered prior to, concurrently with, or subsequent to the administration of an HSP/α2M vaccine composition.

In one embodiment, the HSP preparation is administered to a subject at reasonably the same time as the vaccine, preferably not in admixture. This method provides that the two administrations are performed within a time frame of less than one minute, up to about five minutes, or up to about sixty minutes from each other, for example, at the same doctor's visit.

In one embodiment, the α2M preparation is administered to a subject at reasonably the same time as the vaccine, preferably not in admixture. This method provides that the two administrations are performed within a time frame of less than one minute, up to about five minutes, or up to about sixty minutes from each other, for example, at the same doctor's visit.

In one embodiment, the HSP preparation and HSP/α2M vaccine composition are administered at exactly the same time. In another embodiment the HSP preparation and HSP/α2M vaccine composition are administered in a sequence and within a time interval such that the HSP preparation and HSP/α2M vaccine composition can act together to provide an increased benefit than if they were administered alone. In another embodiment, the HSP preparation and an HSP/α2M vaccine composition are administered sufficiently close in time so as to provide the desired therapeutic or prophylactic outcome. Each can be administered simultaneously or separately, in any appropriate form and by any suitable route. In one embodiment, the HSP preparation and HSP/α2M vaccine composition are administered by different routes of administration. In an alternate embodiment, each is administered by the same route of administration. The HSP preparation can be administered at the same or different sites, e.g. arm and leg. Preferably, when administered simultaneously, the HSP preparation and the HSP/α2M vaccine composition are not administered in admixture or at the same site of administration by the same route of administration.

In one embodiment, the α2M preparation and HSP/α2M vaccine composition are administered at exactly the same time. In another embodiment the α2M preparation and HSP/α2M vaccine composition are administered in a sequence and within a time interval such that the α2M preparation and HSP/α2M vaccine composition can act together to provide an increased benefit than if they were administered alone. In another embodiment, the α2M preparation and an HSP/α2M vaccine composition are administered sufficiently close in time so as to provide the desired therapeutic or prophylactic outcome. Each can be administered simultaneously or separately, in any appropriate form and by any suitable route. In one embodiment, the α2M preparation and HSP/α2M vaccine composition are administered by different routes of administration. In an alternate embodiment, each is administered by the same route of administration. The α2M preparation can be administered at the same or different sites, e.g. arm and leg. Preferably, when administered simultaneously, the α2M preparation and the HSP/α2M vaccine composition are not administered in admixture or at the same site of administration by the same route of administration.

In various embodiments, the HSP preparation and HSP/α2M vaccine composition are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other embodiments, the HSP preparation and HSP/α2M vaccine composition are administered 2 to 4 days apart, 4 to 6 days apart, 1 week a part, 1 to 2 weeks apart, 2 to 4 weeks apart, one moth apart, 1 to 2 months apart, or 2 or more months apart. In preferred embodiments, the HSP preparation and HSP/α2M vaccine composition are administered in a time frame where both are still active. One skilled in the art would be able to determine such a time frame by determining the half life of each administered component.

In various embodiments, the α2M preparation and HSP/α2M vaccine composition are administered less than 1 hour apart, at about 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In other embodiments, the α2M preparation and HSP/α2M vaccine composition are administered 2 to 4 days apart, 4 to 6 days apart, 1 week a part, 1 to 2 weeks apart, 2 to 4 weeks apart, one moth apart, 1 to 2 months apart, or 2 or more months apart. In preferred embodiments, the α2M preparation and HSP/α2M vaccine composition are administered in a time frame where both are still active. One skilled in the art would be able to determine such a time frame by determining the half life of each administered component.

In one embodiment, the HSP preparation and HSP/α2M vaccine composition are administered within the same patient visit. In a specific preferred embodiment, the HSP preparation is administered prior to the administration of the HSP/α2M vaccine composition. In an alternate specific embodiment, the HSP preparation is administered subsequent to the administration of the HSP/α2M vaccine composition.

In one embodiment, the α2M preparation and HSP/α2M vaccine composition are administered within the same patient visit. In a specific preferred embodiment, the α2M preparation is administered prior to the administration of the HSP/α2M vaccine composition. In an alternate specific embodiment, the α2M preparation is administered subsequent to the administration of the HSP/α2M vaccine composition.

In certain embodiments, the HSP preparation or the α2M preparation and HSP/α2M vaccine composition are cyclically administered to a subject. Cycling therapy involves the administration of the HSP preparation or an α2M preparation for a period of time, followed by the administration of an HSP/α2M vaccine composition for a period of time and repeating this sequential administration. Cycling therapy can improve the efficacy of the treatment, reduce the development of resistance to one or more of the therapies, and avoid or reduce the side effects of one of the therapies. In such embodiments, the invention contemplates the alternating administration of an HSP/α2M vaccine composition followed by the administration of an HSP preparation 4 to 6 days later, preferable 2 to 4 days, later, more preferably 1 to 2 days later, wherein such a cycle may be repeated as many times as desired. In certain embodiments, the HSP/α2M vaccine composition and heat shock protein preparation are alternately administered in a cycle of less than 3 weeks, once every two weeks, once every 10 days or once every week. The invention also contemplates the alternating administration of an HSP/α2M vaccine composition followed by the administration of an α2M preparation 4 to 6 days later, preferable 2 to 4 days, later, more preferably 1 to 2 days later, wherein such a cycle may be repeated as many times as desired. In certain embodiments, the HSP/α2M vaccine composition and α2M preparation are alternately administered in a cycle of less than 3 weeks, once every two weeks, once every 10 days or once every week.

In yet another embodiment, the invention provides a method for inducing an immune response by an HSP/α2M vaccine composition in a subject, wherein a sub-immunogenic amount of HSP/α2M vaccine composition is used. As used herein, a sub-immunogenic amount of an HSP/α2M vaccine composition refers to an amount that is insufficient for inducing an immune response if the HSP/α2M vaccine composition is administered independent of the HSP preparation or α2M preparation. The method comprises administering to the subject an amount of a heat shock protein preparation or an amount of an α2M preparation before, concurrently with, or after the administration of the HSP/α2M vaccine composition, such that said amount of HSP/α2M vaccine composition effectively induces an immune response in the subject. In a preferred embodiment, if the component of the HSP/α2M vaccine composition is a peptide complexed to an heat shock protein, the HSP vaccine composition and the heat shock protein preparation are not present in admixture. In yet another preferred embodiment, if the component of the HSP/α2M vaccine composition is a peptide complexed to an α2M, the α2M vaccine composition and the α2M preparation are not present in admixture.

In other embodiments, each of the above embodiments may comprise administration of HSP preparation and α2M preparation in conjunction with an HSP/α2M vaccine composition. In a preferred embodiment, the HSP/α2M vaccine composition is an HSP-peptide complex. In another preferred embodiment, the HSP/α2M vaccine composition is an α2M-peptide complex. In another preferred embodiment, if the component of the HSP/α2M vaccine composition is a peptide complexed to a heat shock protein, the HSP vaccine composition, the heat shock protein preparation and the α2M preparation are not present in admixture.

In various embodiments, the methods of the invention are used to treat or prevent any disease or disorder in which a therapeutic or prophylactic HSP/α2M vaccine composition would be useful, i.e., that is amenable to treatment or prevention by an enhanced immune response. In specific embodiments the disease is an infectious disease or a cancer. The heat shock protein preparation or α2M preparation is generally administered separately from the HSP/α2M vaccine composition.

The invention includes methods for inducing an immune response comprising administering to the subject an HSP/α2M vaccine composition comprising an HSP or α2M complexed to a component against which an immune response is desired to be induced; and administering to the subject a heat shock protein preparation, wherein the heat shock protein preparation does not elicit an immune response against the component in the absence of the administering of the HSP/α2M vaccine composition. In a preferred embodiment, the HSP/α2M vaccine composition does not comprise a heat shock protein or an α2M. In another preferred embodiment, if the component of the HSP/α2M vaccine composition is a peptide complexed to an heat shock protein, the HSP vaccine composition and the heat shock protein preparation are not present in admixture. In yet another preferred embodiment, if the component of the HSP/α2M vaccine composition is a peptide complexed to an α2M, the α2M vaccine composition and the α2M preparation are not present in admixture.

The invention includes methods for inducing an immune response comprising administering to the subject an HSP/α2M vaccine composition comprising an HSP or α2M complexed to a component against which an immune response is desired to be induced; and administering to the subject an α2M preparation, wherein the α2M preparation does not elicit an immune response against the component in the absence of the administering of the HSP/α2M vaccine composition. In another preferred embodiment, if the component of the HSP/α2M vaccine composition is a peptide complexed to an heat shock protein, the HSP vaccine composition and the heat shock protein preparation are not present in admixture. In yet another preferred embodiment, if the component of the HSP/α2M vaccine composition is a peptide complexed to an α2M, the α2M vaccine composition and the α2M preparation are not present in admixture.

The invention encompasses methods for treating or preventing an infectious disease in a subject comprising in any order the steps of administering to the subject an HSP/α2M vaccine composition comprising an HSP or α2M complexed to a component that displays the antigenicity of an infectious agent that causes the infectious disease (e.g., an immunogenic amount of an antigen on the causative infectious agent); and administering to the subject an amount of a heat shock protein preparation effective in combination with the HSP/α2M vaccine composition to induce or increase an immune response to the component in the subject, wherein the heat shock protein preparation does not elicit an immune response against said component in the absence of said administering of the HSP/α2M vaccine composition. In another specific embodiment, if the component of the HSP/α2M vaccine composition is a peptide complexed to an heat shock protein, the HSP vaccine composition and the heat shock protein preparation are not present in admixture. In yet another specific embodiment, if the component of the HSP/α2M vaccine composition is a peptide complexed to an α2M, the α2M vaccine composition and the α2M preparation are not present in admixture.

The invention encompasses methods for treating or preventing an infectious disease in a subject comprising in any order the steps of administering to the subject an HSP/α2M vaccine composition comprising an HSP or α2M complexed to a component that displays the antigenicity of an infectious agent that causes the infectious disease (e.g., an immunogenic amount of an antigen on the causative infectious agent); and administering to the subject an amount of an α2M preparation effective in combination with the HSP/α2M vaccine composition to induce or increase an immune response to the component in the subject, wherein the α2M preparation does not elicit an immune response against said component in the absence of said administering of the HSP/α2M vaccine composition. In another specific embodiment, if the component of the HSP/α2M vaccine composition is a peptide complexed to an heat shock protein, the HSP vaccine composition and the heat shock protein preparation are not present in admixture. In yet another specific embodiment, if the component of the HSP/α2M vaccine composition is a peptide complexed to an α2M, the α2M vaccine composition and the α2M preparation are not present in admixture.

The invention also encompasses methods for treating or preventing a cancer or metastasis in a subject comprising in any order the steps of administering to the subject an HSP/α2M vaccine composition comprising an HSP or α2M complexed to a component that displays the antigenicity of a cancer cell (e.g., an immunogenic amount of an antigen on a cancer, such as but not limited to a tumor-specific antigen, and a tumor-associated antigen, or a molecule displaying antigenicity thereof); and administering to the subject an amount of a heat shock protein preparation effective to induce or increase an immune response in the subject to the component, wherein the heat shock protein preparation does not elicit an immune response against the component in the absence of the administering of the HSP/α2M vaccine composition. Preferably, the component of the HSP/α2M vaccine composition and the heat shock protein preparation are not present in admixture.

The invention also encompasses methods for treating or preventing a cancer or metastasis in a subject comprising in any order the steps of administering to the subject an HSP/α2M vaccine composition comprising an HSP or α2M complexed to a component that displays the antigenicity of a cancer cell (e.g., an immunogenic amount of an antigen on a cancer, such as but not limited to a tumor-specific antigen, and a tumor-associated antigen, or a molecule displaying antigenicity thereof); and administering to the subject an amount of an α2M preparation effective to induce or increase an immune response in the subject to the component, wherein the α2M preparation does not elicit an immune response against the component in the absence of the administering of the HSP/α2M vaccine composition. Preferably, the component of the HSP/α2M vaccine composition and the heat shock protein preparation are not present in admixture.

Where HSP-peptide complexes or α2M-peptide complexes are used as an HSP or α2M preparation, the peptides preferably do not display the antigenicity of a molecule/component of the HSP/α2M vaccine composition. In this instance, the purpose of the invention is not to use an HSP-peptide complex or an α2M-peptide complex to elicit a specific immune response against a peptide present in the HSP/α2M vaccine composition. The HSP preparations and the α2M preparations of the invention generally aid presentation of all kinds of antigens in the subject, particularly those administered to the subject in the HSP/α2M vaccine composition.

Where HSP-peptide complexes or α2M-peptide complexes are used as HSP/α2M vaccine compositions, the peptides display the antigenicity of a molecule/component relevant to the condition in question (“relevant” meaning that an immune response thereto would be therapeutic or prophylactic for the condition in question).

4.1. Preparation of Heat Shock Proteins

Three major families of HSPs have been identified based on molecular weight. The families have been called hsp60, hsp70 and hsp90, where the numbers reflect the approximate molecular weight of the stress proteins in kilodaltons. Many members of these families were found subsequently to be induced in response to other stressful stimuli including, but not limited to, nutrient deprivation, metabolic disruption, oxygen radicals and infection with intracellular pathogens (See Welch, May 1993, Scientific American 56-64; Young, 1990, Annu. Rev. Immunol. 8:401-420; Craig, 1993, Science 260:1902-1903; Gething, et al., 1992, Nature 355:33-45; and Lindquist, et al., 1988, Annu. Rev. Genetics 22:631-677). A number of proteins thought to be involved in chaperoning functions are residents of the endoplasmic reticulum (ER) lumen and include, for example, protein disulfide isomerase (PDI; Gething et al., 1992, Nature 355:33-45), calreticulin (Herbert et al., 1997, J. Cell Biol. 139:613-623), Grp94 or ERp99 (Sorger & Pelham, 1987, J. Mol. Biol. 194:(2) 341-4) which is related to hsp90, and Grp78 or BiP, which is related to hsp70 (Munro et al., 1986, Cell 46:291-300; Haas & Webl, 1983, Nature 306:387-389). It is contemplated that HSPs belonging to all of these three families, including fragments of such HSPs, can be used in the practice of the instant invention.

This invention also contemplates the use of other HSPs, and fragments thereof, including but not limited to hsp110, gp96, grp170 and calreticulin.

Heat shock proteins are among the most highly conserved proteins in existence. For example, DnaK, the hsp70 from E. coli has about 50% amino acid sequence identity with hsp70 proteins from excoriates (Bardwell, et al., 1984, Proc. Natl. Acad. Sci. 81:848-852). The hsp60 and hsp90 families also show similarly high levels of intra families conservation (Hickey, et al., 1989, Mol. Cell. Biol. 9:2615-2626; Jindal, 1989, Mol. Cell. Biol. 9:2279-2283). In addition, it has been discovered that the hsp60, hsp70 and hsp90 families are composed of proteins that are related to the stress proteins in sequence, for example, having greater than 35% amino acid identity, but whose expression levels are not altered by stress. Therefore it is contemplated that the definition of stress protein, as used herein, embraces other proteins, muteins, analogs, and variants thereof having at least 35% to 55%, preferably 55% to 75%, and most preferably 75% to 85% amino acid identity with members of the three families whose expression levels in a cell are enhanced in response to a stressful stimulus. The purification of stress proteins belonging to these three families is described below.

In addition, HSPs have been found to have immunological and antigenic properties. HSPs are now understood to play an essential role in immune regulation. For instance, prior experiments have demonstrated that HSPs stimulate strong and long-lasting specific immune responses against antigenic peptides that have been covalently or noncovalently attached to the HSPs. By utilizing a specific peptide, the immune response generated is “specific” or targeted to that peptide.

In the present invention, purified unbound HSPs, HSPs covalently or noncovalently bound to specific peptides or nonspecific peptides (collectively referred to herein as HSP-peptide complexes), and combinations of thereof are used. Purification of HSPs in complexed or non-complexed forms are described in the following subsections. Further, one skilled in the art can synthesize HSPs by recombinant expression or peptide synthesis, which are also described below. In the present invention, an HSP preparation can comprise unbound hsp70, hsp90, gp96, calreticulin, hsp110 or grp170 or noncovalent or covalent complexes thereof complexed to a peptide. Methods of preparation and purification of HSPs are known in the art and described below. More specifically, methods of preparing and purifying calreticulin and cellular, non-covalently bound calreticulin peptide-complexes are known in the art. Literature such as Basu and Srivastava, 1999, J. Exp. Med. 189:797-802, describes such methods and is hereby incorporated by reference in its entirety. The purification of other exemplary HSPs and HSP-peptide complexes is described in detail below.

4.1.1. Preparation and Purification of HSP70 and Cellular, Non-Covalently Bound HSP70-Peptide Complexes

The purification of hsp70-peptide complexes has been described previously, see, for example, Udono et al., 1993, J. Exp. Med. 178:1391-1396. A procedure that may be used, presented by way of example but not limitation, is as follows:

Initially, human or mammalian cells are suspended in 3 volumes of 1×Lysis buffer consisting of 5 mM sodium phosphate buffer (pH 7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate (pH 7.5), 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose™ equilibrated with phosphate buffered saline (PBS) containing 2 mM Ca²⁺ and 2 mM Mg²⁺. When the cells are lysed by mechanical shearing the supernatant is diluted with an equal volume of 2× lysis buffer prior to mixing with Con A Sepharose™. The supernatant is then allowed to bind to the Con A Sepharose™ for 2-3 hours at 4° C. The material that fails to bind is harvested and dialyzed for 36 hours (three times, 100 volumes each time) against 100 mM Tris-Acetate (pH 7.5), 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC™ ion exchange chromatographic column (Pharmacia) equilibrated in 20 mM Tris-Acetate (pH 7.5), 20 mM NaCl, 0.1 mM EDTA and 15 mM 2-mercaptoethanol. The column is then developed with a 20 mM to 500 mM NaCl gradient and then eluted fractions fractionated by sodium dodecyl sulfate-polyacrylamide gel electrophoresis (SDS-PAGE) and characterized by immunoblotting using an appropriate anti-hsp70 antibody (such as from clone N27F3-4, from StressGen).

Fractions strongly immunoreactive with the anti-hsp70 antibody are pooled and the hsp70-peptide complexes precipitated with ammonium sulfate; specifically with a 50%-70% ammonium sulfate cut. The resulting precipitate is then harvested by centrifugation at 17,000 rpm (SS34 Sorvall rotor) and washed with 70% ammonium sulfate. The washed precipitate is then solubilized and any residual ammonium sulfate removed by gel filtration on a Sephadex® G25 column (Pharmacia). If necessary the hsp70 preparation thus obtained can be repurified through the Mono Q FPLC™ ion exchange chromatographic column (Pharmacia) as described above.

The hsp70-peptide complex can be purified to apparent homogeneity using this method. Typically 1 mg of hsp70-peptide complex can be purified from 1 g of cells/tissue.

An improved method for purification of hsp70-peptide complexes comprises contacting cellular proteins with ADP or a nonhydrolyzable analog of ATP affixed to a solid substrate, such that hsp70 in the lysate can bind to the ADP or nonhydrolyzable ATP analog, and eluting the bound hsp70. A preferred method uses column chromatography with ADP affixed to a solid substratum (e.g., ADP-agarose). The resulting hsp70 preparations are higher in purity and devoid of contaminating peptides. The hsp70 complex yields are also increased significantly by about more than 10 fold. Alternatively, chromatography with nonhydrolyzable analogs of ATP, instead of ADP, can be used for purification of hsp70-peptide complexes. By way of example but not limitation, purification of hsp70-peptide complexes by ADP-agarose chromatography can be carried out as follows:

Meth A sarcoma cells (500 million cells) are homogenized in hypotonic buffer and the lysate is centrifuged at 100,000 g for 90 minutes at 4° C. The supernatant is applied to an ADP-agarose column. The column is washed in buffer and is eluted with 5 column volumes of 3 mM ADP. The hsp70-peptide complexes elute in fractions 2 through 10 of the total 15 fractions which elute. The eluted fractions are analyzed by SDS-PAGE. The hsp70-peptide complexes can be purified to apparent homogeneity using this procedure.

Separation of the HSP from an hsp70-peptide complex can be performed in the presence of ATP or low pH. These two methods may be used to elute the peptide from an hsp70-peptide complex. The first approach involves incubating an hsp70-peptide complex preparation in the presence of ATP. The other approach involves incubating an hsp70-peptide complex preparation in a low pH buffer. These methods and any others known in the art may be applied to separate the HSP and peptide from an hsp-peptide complex.

4.1.2. Preparation and Purification of HSP90 and Cellular, Non-covalently Bound HSP90-Peptide complexes

A procedure that can be used, presented by way of example and not limitation, is as follows:

Initially, human or mammalian cells are suspended in 3 volumes of 1×Lysis buffer consisting of 5 mM sodium phosphate buffer (pH 7), 150 mM NaCl, 2 mM CaCl₂, 2 mM MgCl₂ and 1 mM phenyl methyl sulfonyl fluoride (PMSF). Then, the pellet is sonicated, on ice, until >99% cells are lysed as determined by microscopic examination. As an alternative to sonication, the cells may be lysed by mechanical shearing and in this approach the cells typically are resuspended in 30 mM sodium bicarbonate (pH 7.5), 1 mM PMSF, incubated on ice for 20 minutes and then homogenized in a Dounce homogenizer until >95% cells are lysed.

Then the lysate is centrifuged at 1,000 g for 10 minutes to remove unbroken cells, nuclei and other cellular debris. The resulting supernatant is recentrifuged at 100,000 g for 90 minutes, the supernatant harvested and then mixed with Con A Sepharose™ equilibrated with PBS containing 2 mM Ca²⁺ and 2 mM Mg²⁺. When the cells are lysed by mechanical shearing the supernatant is diluted with an equal volume of 2× Lysis buffer prior to mixing with Con A Sepharose™. The supernatant is then allowed to bind to the Con A Sepharose™ for 2-3 hours at 4° C. The material that fails to bind is harvested and dialyzed for 36 hours (three times, 100 volumes each time) against 10 mM Tris-Acetate (pH 7.5), 0.1 mM EDTA, 10 mM NaCl, 1 mM PMSF. Then the dialyzate is centrifuged at 17,000 rpm (Sorvall SS34 rotor) for 20 minutes. Then the resulting supernatant is harvested and applied to a Mono Q FPLC™ ion exchange chromatographic column (Pharmacia) equilibrated with lysis buffer. The proteins are then eluted with a salt gradient of 200 mM to 600 mM NaCl.

The eluted fractions are fractionated by SDS-PAGE and fractions containing the hsp90-peptide complexes identified by immunoblotting using an anti-hsp90 antibody such as 3G3 (Affinity Bioreagents). Hsp90-peptide complexes can be purified to apparent homogeneity using this procedure. Typically, 150-200 μg of hsp90-peptide complex can be purified from 1 g of cells/tissue.

Separation of the HSP from an hsp90-peptide complex can be performed in the presence of ATP or low pH. These two methods may be used to elute the peptide from an hsp90-peptide complex. The first approach involves incubating an hsp90-peptide complex preparation in the presence of ATP. The other approach involves incubating an hsp90-peptide complex preparation in a low pH buffer. These methods and any others known in the art may be applied to separate the HSP and peptide from an hsp-peptide complex.

4.1.3. Preparation and Purification of GP96 and Cellular, Non-Covalently Bound GP96-Peptide Complexes

A procedure that can be used, presented by way of example and not limitation, is as follows:

A pellet of human or mammalian cells is resuspended in 3 volumes of buffer consisting of 30 mM sodium bicarbonate buffer (pH 7.5) and 1 mM PMSF and the cells allowed to swell on ice 20 minutes. The cell pellet is then homogenized in a Dounce homogenizer (the appropriate clearance of the homogenizer will vary according to each cell type) on ice until >95% cells are lysed.

The lysate is centrifuged at 1,000 g for 10 minutes to remove unbroken cells, nuclei and other debris. The supernatant from this centrifugation step is then recentrifuged at 100,000 g for 90 minutes. The gp96-peptide complex can be purified either from the 100,000 pellet or from the supernatant.

When purified from the supernatant, the supernatant is diluted with equal volume of 2× lysis buffer and the supernatant mixed for 2-3 hours at 4° C. with Con A Sepharose™ equilibrated with PBS containing 2 mM Ca²⁺ and 2 mM Mg²⁺. Then, the slurry is packed into a column and washed with 1× lysis buffer until the OD₂₈₀ drops to baseline. Then, the column is washed with ⅓ column bed volume of 10% α-methyl mannoside (α-MM) dissolved in PBS containing 2 mM Ca²⁺ and 2 mM Mg²⁺, the column sealed with a piece of parafilm, and incubated at 37° C. for 15 minutes. Then the column is cooled to room temperature and the parafilm removed from the bottom of the column. Five column volumes of the α-MM buffer are applied to the column and the eluate analyzed by SDS-PAGE. Typically the resulting material is about 60-95% pure, however this depends upon the cell type and the tissue-to-lysis buffer ratio used. Then the sample is applied to a Mono Q FPLC™ ion exchange chromatographic column (Pharmacia) equilibrated with a buffer containing 5 mM sodium phosphate (pH 7). The proteins are then eluted from the column with a 0-1M NaCl gradient and the gp96 fraction elutes between 400 mM and 550 mM NaCl.

The procedure, however, may be modified by two additional steps, used either alone or in combination, to consistently produce apparently homogeneous gp96-peptide complexes. One optional step involves an ammonium sulfate precipitation prior to the Con A purification step and the other optional step involves DEAE-Sepharose™ purification after the Con A purification step but before the Mono Q FPLC™ step.

In the first optional step, described by way of example as follows, the supernatant resulting from the 100,000 g centrifugation step is brought to a final concentration of 50% ammonium sulfate by the addition of ammonium sulfate. The ammonium sulfate is added slowly while gently stirring the solution in a beaker placed in a tray of ice water. The solution is stirred from about ½ to 12 hours at 4° C. and the resulting solution centrifuged at 6,000 rpm (Sorvall SS34 rotor). The supernatant resulting from this step is removed, brought to 70% ammonium sulfate saturation by the addition of ammonium sulfate solution, and centrifuged at 6,000 rpm (Sorvall SS34 rotor). The resulting pellet from this step is harvested and suspended in PBS containing 70% ammonium sulfate in order to rinse the pellet. This mixture is centrifuged at 6,000 rpm (Sorvall SS34 rotor) and the pellet dissolved in PBS containing 2 mM Ca²⁺ and Mg²⁺. Undissolved material is removed by a brief centrifugation at 15,000 rpm (Sorvall SS34 rotor). Then, the solution is mixed with Con A Sepharose™ and the procedure followed as before.

In the second optional step, described by way of example as follows, the gp96 containing fractions eluted from the Con A column are pooled and the buffer exchanged for 5 mM sodium phosphate buffer (pH 7), 300 mM NaCl by dialysis, or preferably by buffer exchange on a Sephadex G25 column. After buffer exchange, the solution is mixed with DEAE-Sepharose™ previously equilibrated with 5 mM sodium phosphate buffer (pH 7), 300 mM NaCl. The protein solution and the beads are mixed gently for 1 hour and poured into a column. Then, the column is washed with 5 mM sodium phosphate buffer (pH 7), 300 mM NaCl, until the absorbance at 280 nm drops to baseline. Then, the bound protein is eluted from the column with five volumes of 5 mM sodium phosphate buffer (pH 7), 700 mM NaCl. Protein containing fractions are pooled and diluted with 5 mM sodium phosphate buffer (pH 7) in order to lower the salt concentration to 175 mM. The resulting material then is applied to the Mono Q FPLC™ ion exchange chromatographic column (Pharmacia) equilibrated with 5 mM sodium phosphate buffer (pH 7) and the protein that binds to the Mono Q FPLC™ ion exchange chromatographic column (Pharmacia) is eluted as described before.

It is appreciated, however, that one skilled in the art may assess, by routine experimentation, the benefit of incorporating the second optional step into the purification protocol. In addition, it is appreciated also that the benefit of adding each of the optional steps will depend upon the source of the starting material.

When the gp96 fraction is isolated from the 100,000 g pellet, the pellet is suspended in 5 volumes of PBS containing either 1% sodium deoxycholate or 1% oxtyl glucopyranoside (but without the Mg²⁺ and Ca²⁺) and incubated on ice for 1 hour. The suspension is centrifuged at 20,000 g for 30 minutes and the resulting supernatant dialyzed against several changes of PBS (also without the Mg²⁺ and Ca²⁺) to remove the detergent. The dialysate is centrifuged at 100,000 g for 90 minutes, the supernatant harvested, and calcium and magnesium are added to the supernatant to give final concentrations of 2 mM, respectively. Then the sample is purified by either the unmodified or the modified method for isolating gp96-peptide complex from the 100,000 g supernatant, see above.

The gp96-peptide complexes can be purified to apparent homogeneity using this procedure. About 10-20 μg of gp96 can be isolated from 1 g cells/tissue.

Separation of the HSP from an gp96-peptide complex can be performed in the presence of ATP or low pH. These two methods may be used to elute the peptide from an gp96-peptide complex. The first approach involves incubating an gp96-peptide complex preparation in the presence of ATP. The other approach involves incubating an gp96-peptide complex preparation in a low pH buffer. These methods and any others known in the art may be applied to separate the HSP and peptide from an hsp-peptide complex.

4.1.4. Preparation and Purification of HSP110 and Cellular, Non-Covalently Bound HSP110-Peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose unbound material is first dialyzed against a solution of 20 mM Tris-HCl, pH 7.5; 100 mM NaCl; and 15 mM 2-ME, and then applied to a DEAE-Sepharose column and eluted by salt gradient from 100 to 500 mM NaCl. Fractions containing hsp110 are collected, dialyzed, and loaded onto a Mono Q (Pharmacia) 10/10 column equilibrated with 20 mM Tris-HCl, pH 7.5; 200 mM NaCl; and 15 mM 2-ME. The bound proteins are eluted with a 200-500 mM NaCl gradient. Fractions are analyzed by SDS-PAGE followed by immunoblotting with an Ab for hsp110, as described by Wang et al., 1999, J. Immunol. 162:3378. Pooled fractions containing hsp110 are concentrated by Centriplus (Amicon, Beverly, Mass.) and applied to a Superose 12 column (Pharmacia). Proteins are eluted by 40 mM Tris-HCl, pH 8.0; 150 mM NaCl; and 15 mM 2-ME with a flow rate of 0.2 ml/min.

4.1.5. Preparation and Purification of GRP170 and Cellular, Non-Covalently Bound GRP170-Peptide Complexes

A procedure, described by Wang et al., 2001, J. Immunol. 166(1):490-7, that can be used, presented by way of example and not limitation, is as follows:

A pellet (40-60 ml) of cell or tissue, e.g., tumor cell tissue, is homogenized in 5 vol of hypotonic buffer (30 mN sodium bicarbonate, pH7.2, and protease inhibitors) by Dounce homogenization. The lysate is centrifuged at 4,500×g and then 100,000×g for 2 hours. If the cells or tissues are of hepatic origin, the resulting supernatant is was first applied to a blue Sepharose column (Pharmacia) to remove albumin. Otherwise, the resulting supernatant is applied to a Con A-Sepharose column (Pharmacia Biotech, Piscataway, N.J.) previously equilibrated with binding buffer (20 mM Tris-HCI, pH 7.5; 100 mM NaCl; 1 mM MgCl₂; 1 mM CaCl₂; 1 mM MnCl₂; and 15 mM 2-ME). The bound proteins are eluted with binding buffer containing 15% α-D-o-methylmannoside (Sigma, St. Louis, Mo.).

Con A-Sepharose-bound material is first dialyzed against 20 mM Tris-HCl, pH 7.5, and 150 mM NaCl and then applied to a Mono Q column and eluted by a 150 to 400 mM NaCl gradient. Pooled fractions are concentrated and applied on the Superose 12 column (Pharmacia). Fractions containing homogeneous grp170 are collected.

4.1.6. Recombinany Expression of HSPS

Methods known in the art can be utilized to recombinantly produce HSPs. A nucleic acid sequence encoding a heat shock protein can be inserted into an expression vector for propagation and expression in host cells.

An expression construct, as used herein, refers to a nucleotide sequence encoding an HSP operably associated with one or more regulatory regions which enables expression of the HSP in an appropriate host cell. “Operably-associated” refers to an association in which the regulatory regions and the HSP sequence to be expressed are joined and positioned in such a way as to permit transcription, and ultimately, translation.

The regulatory regions necessary for transcription of the HSP can be provided by the expression vector. A translation initiation codon (ATG) may also be provided if the HSP gene sequence lacking its cognate initiation codon is to be expressed. In a compatible host-construct system, cellular transcriptional factors, such as RNA polymerase, will bind to the regulatory regions on the expression construct to effect transcription of the modified HSP sequence in the host organism. The precise nature of the regulatory regions needed for gene expression may vary from host cell to host cell. Generally, a promoter is required which is capable of binding RNA polymerase and promoting the transcription of an operably-associated nucleic acid sequence. Such regulatory regions may include those 5′ non-coding sequences involved with initiation of transcription and translation, such as the TATA box, capping sequence, CAAT sequence, and the like. The non-coding region 3′ to the coding sequence may contain transcriptional termination regulatory sequences, such as terminators and polyadenylation sites.

In order to attach DNA sequences with regulatory functions, such as promoters, to the HSP gene sequence or to insert the HSP gene sequence into the cloning site of a vector, linkers or adapters providing the appropriate compatible restriction sites may be ligated to the ends of the cDNAs by techniques well known in the art (Wu et al., 1987, Methods in Enzymol 152:343-349). Cleavage with a restriction enzyme can be followed by modification to create blunt ends by digesting back or filling in single-stranded DNA termini before ligation. Alternatively, a desired restriction enzyme site can be introduced into a fragment of DNA by amplification of the DNA by use of PCR with primers containing the desired restriction enzyme site.

An expression construct comprising an HSP sequence operably associated with regulatory regions can be directly introduced into appropriate host cells for expression and production of HSP-peptide complexes without further cloning. See, for example, U.S. Pat. No. 5,580,859. The expression constructs can also contain DNA sequences that facilitate integration of the HSP sequence into the genome of the host cell, e.g., via homologous recombination. In this instance, it is not necessary to employ an expression vector comprising a replication origin suitable for appropriate host cells in order to propagate and express the HSP in the host cells.

A variety of expression vectors may be used including, but not limited to, plasmids, cosmids, phage, phagemids or modified viruses. Typically, such expression vectors comprise a functional origin of replication for propagation of the vector in an appropriate host cell, one or more restriction endonuclease sites for insertion of the HSP gene sequence, and one or more selection markers. The expression vector must be used with a compatible host cell which may be derived from a prokaryotic or an eukaryotic organism including but not limited to bacteria, yeasts, insects, mammals and humans.

For long term, high yield production of properly processed HSP or HSP-peptide complexes, stable expression in mammalian cells is preferred. Cell lines that stably express HSP or HSP-peptide complexes may be engineered by using a vector that contains a selectable marker. By way of example but not limitation, following the introduction of the expression constructs, engineered cells may be allowed to grow for 1-2 days in an enriched media, and then are switched to a selective media. The selectable marker in the expression construct confers resistance to the selection and optimally allows cells to stably integrate the expression construct into their chromosomes and to grow in culture and to be expanded into cell lines. Such cells can be cultured for a long period of time while HSP is expressed continuously.

The recombinant cells may be cultured under standard conditions of temperature, incubation time, optical density and media composition. However, conditions for growth of recombinant cells may be different from those for expression of HSPs and antigenic proteins. Modified culture conditions and media may also be used to enhance production of the HSP. For example, recombinant cells containing HSPs with their cognate promoters may be exposed to heat or other environmental stress, or chemical stress. Any techniques known in the art may be applied to establish the optimal conditions for producing HSP or HSP-peptide complexes.

4.1.7. Peptide Synthesis

An alternative to producing HSP by recombinant techniques is peptide synthesis. For example, an entire HSP, or a peptide corresponding to a portion of an HSP can be synthesized by use of a peptide synthesizer. Conventional peptide synthesis or other synthetic protocols well known in the art may be used.

Peptides having the amino acid sequence of an HSP or a portion thereof may be synthesized by solid-phase peptide synthesis using procedures similar to those described by Merrifield, 1963, J. Am. Chem. Soc., 85:2149. During synthesis, N-α-protected amino acids having protected side chains are added stepwise to a growing polypeptide chain linked by its C-terminal and to an insoluble polymeric support i.e., polystyrene beads. The peptides are synthesized by linking an amino group of an N-α-deprotected amino acid to an α-carboxyl group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc which is acid labile and Fmoc which is base labile. Details of appropriate chemistries, resins, protecting groups, protected amino acids and reagents are well known in the art and so are not discussed in detail herein (See, Atherton, et al., 1989, Solid Phase Peptide Synthesis: A Practical Approach, IRL Press, and Bodanszky, 1993, Peptide Chemistry, A Practical Textbook, 2nd Ed., Springer-Verlag).

Purification of the resulting HSP is accomplished using conventional procedures, such as preparative HPLC using gel permeation, partition and/or ion exchange chromatography. The choice of appropriate matrices and buffers are well known in the art and so are not described in detail herein.

4.2. Preparation of α2M

Alpha-2-macroglobulin (α2M) can be bought from commercial sources or prepared by purifying it from human blood. To purify α2M-peptide complexes from blood, the following non-limiting protocol can be used:

Blood is collected from a subject and is allowed to clot. It is then centrifuged for 30 minutes under 14,000×g to obtain the serum which is then applied to a gel filtration column (Sephacryl S-300R) equilibrated with 0.04M Tris buffer pH 7.6 plus 0.3M NaCl. A 65 ml column is used for about 10 ml of serum. Three ml fractions are collected and each fraction is tested for the presence of α2M by dot blot using an α2M specific antibody. The α2M positive fractions are pooled and applied to a PD10 column to exchange the buffer to 0.1M Sodium Phosphate buffer pH 7.5 with PMSF. The pooled fractions are then applied to a Con A column (10 ml) equilbrated with the phosphate buffer. The column is washed and the protein is eluted with 5% methylmannose pyranoside. The eluent is passed over a PD10 column to change the buffer to a Sodium Acetate buffer (0.05M; pH6.0). A DEAE column is then equilibrated with acetate buffer and the sample is applied to the DEAE column. The column is washed and the protein is eluted with 0.13M sodium acetate. The fractions with α2M are then pooled.

4.2.1. Recombinant Expression of Heat Shock Proteins and α2M

In certain embodiments of the present invention, HSPs and α2M can be prepared from cells that express higher levels of HSPs and α2M through recombinant means. Amino acid sequences and nucleotide sequences of many HSPs and α2M are generally available in sequence databases, such as GenBank. Computer programs, such as Entrez, can be used to browse the database, and retrieve any amino acid sequence and genetic sequence data of interest by accession number. These databases can also be searched to identify sequences with various degrees of similarities to a query sequence using programs, such as FASTA and BLAST, which rank the similar sequences by alignment scores and statistics. Such nucleotide sequences of non-limiting examples of HSPs that can be used for the compositions, methods, and for preparation of the HSP peptide-complexs of the invention are as follows: human HSP70, Genbank Accession No.M24743, Hunt et al., 1995, Proc. Natl. Acad. Sci. U.S.A., 82: 6455-6489; human HSP90, Genbank Accession No. X15183, Yamazaki et al., Nucl. Acids Res. 17: 7108; human gp96: Genbank Accession No. X15187, Maki et al., 1990, Proc. Natl. Acad. Sci. U.S.A. 87: 5658-5562; human BiP: Genbank Accession No.M19645; Ting et al., 1988, DNA 7: 275-286; human HSP27, Genbank Accession No.M24743; Hickey et al., 1986, Nucleic Acids Res. 14: 4127-45; mouse HSP70: Genbank Accession No.M35021, Hunt et al., 1990, Gene 87: 199-204; mouse gp96: Genbank Accession No.M16370, Srivastava et al., 1987, Proc. Natl. Acad. Sci. U.S.A. 85: 3807-3811; and mouse BiP: Genbank Accession No.U16277, Haas et al., 1988, Proc. Natl. Acad. Sci. U.S.A. 85: 2250-2254. Due to the degeneracy of the genetic code, not only the naturally occurring nucleotide sequence but also all the other degenerate DNA sequences that encode the HSP can be used to express HSP.

In addition and alternatively to the α2M protein, polypeptide fragments, analogs, and variants of α2M can also be used in the practice of the claimed invention, that have at least 35% to 55%, preferably 55% to 75%, and most preferably 75% to 85% amino acid identity with α2M, and is capable of forming a complex with an antigenic peptide, which complex is capable of being taken up by an antigen presenting cell and eliciting an immune response against the antigenic molecule. The α2M molecule of the invention can be purchased commercially or purified from natural sources (Kurecki et al., 1979, Anal. Biochem. 99:415-420), chemically synthesized, or recombinantly produced. Non-limiting examples of α2M sequences that can be used for preparation of the α2M polypeptides of the invention are as follows: Genbank Accession Nos. M11313, P01023, AAA51551; Kan et al., 1985, Proc. Nat. Acad. Sci. 82: 2282-2286. Due to the degeneracy of the genetic code, not only the naturally occurring nucleotide sequence but also all the other degenerate DNA sequences that encode the α2M can also be used to express α2M.

Once the nucleotide sequence encoding the HSP or α2M of choice has been identified, the nucleotide sequence, or a fragment thereof, can be obtained and cloned into an expression vector for recombinant expression. The expression vector can then be introduced into a host cell for propogation of the HSP or α2M. Methods for recombinant production of HSPs or α2M are described in detail herein.

The DNA may be obtained by DNA amplification or molecular cloning directly from a tissue, cell culture, or cloned DNA (e.g., a DNA “library”) using standard molecular biology techniques (see e.g., Methods in Enzymology, 1987, volume 154, Academic Press; Sambrook et al. 1989, Molecular Cloning—A Laboratory Manual, 2nd Edition, Cold Spring Harbor Press, New York; and Current Protocols in Molecular Biology, Ausubel et al. (eds.), Greene Publishing Associates and Wiley Interscience, New York, each of which is incorporated herein by reference in its entirety). Clones derived from genomic DNA may contain regulatory and intron DNA regions in addition to coding regions; clones derived from cDNA will contain only exon sequences. Whatever the source, the HSP or α2M gene should be cloned into a suitable vector for propagation of the gene.

In a preferred embodiment, DNA can be amplified from genomic or cDNA by polymerase chain reaction (PCR) amplification using primers designed from the known sequence of a related or homologous HSP or α2M. PCR is used to amplify the desired sequence in DNA clone or a genomic or cDNA library, prior to selection. PCR can be carried out, e.g., by use of a thermal cycler and Taq polymerase (Gene Amp®). The polymerase chain reaction (PCR) is commonly used for obtaining genes or gene fragments of interest. For example, a nucleotide sequence encoding an HSP or α2M of any desired length can be generated using PCR primers that flank the nucleotide sequence encoding open reading fram. Alternatively, an HSP or α2M gene sequence can be cleaved at appropriate sites with restriction endonuclease(s) if such sites are available, releasing a fragment of DNA encoding the HSP or α2M gene. If convenient restriction sites are not available, they may be created in the appropriate positions by site-directed mutagenesis and/or DNA amplification methods known in the art (see, for example, Shankarappa et al., 1992, PCR Method Appl. 1: 277-278). The DNA fragment that encodes the HSP or α2M is then isolated, and ligated into an appropriate expression vector, care being taken to ensure that the proper translation reading frame is maintained.

In an alternative embodiment, for the molecular cloning of an HSP or α2M gene from genomic DNA, DNA fragments are generated to form a genomic library. Since some of the sequences encoding related HSPs or α2M are available and can be purified and labeled, the cloned DNA fragments in the genomic DNA library may be screened by nucleic acid hybridization to a labeled probe (Benton and Davis, 1977, Science 196: 180; Grunstein and Hogness, 1975, Proc. Natl. Acad. Sci. U.S.A. 72: 3961). Those DNA fragments with substantial homology to the probe will hybridize. It is also possible to identify an appropriate fragment by restriction enzyme digestion(s) and comparison of fragment sizes with those expected according to a known restriction map.

Alternatives to isolating the HSP or α2M genomic DNA include, but are not limited to, chemically synthesizing the gene sequence itself from a known sequence or synthesizing a cDNA to the mRNA which encodes the HSP or α2M. For example, RNA for cDNA cloning of the HSP or α2M gene can be isolated from cells which express the HSP or α2M. A cDNA library may be generated by methods known in the art and screened by methods, such as those disclosed for screening a genomic DNA library. If an antibody to the HSP or α2M is available, the HSP or α2M may be identified by binding of a labeled antibody to the HSP- or α2M-synthesizing clones.

Other specific embodiments for the cloning of a nucleotide sequence encoding an HSP or α2M, are presented as examples but not by way of limitation, as follows: In a specific embodiment, nucleotide sequences encoding an HSP or α2M can be identified and obtained by hybridization with a probe comprising a nucleotide sequence encoding HSP or α2M under conditions of low to medium stringency. By way of example and not limitation, procedures using such conditions of low stringency are as follows (see also Shilo and Weinberg, 1981, Proc. Natl. Acad. Sci. U.S.A. 78: 6789-6792). Filters containing DNA are pretreated for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Hybridizations are carried out in the same solution with the following modifications: 0.02% PVP, 0.02% Ficoll, 0.2% BSA, 100 μg/ml salmon sperm DNA, 10% (wt/vol) dextran sulfate, and 5-20×10⁶ cpm ³²P-labeled probe is used. Filters are incubated in hybridization mixture for 18-20 h at 40° C., and then washed for 1.5 h at 55° C. in a solution containing 2×SSC, 25 mM Tris-HCl (pH 7.4), 5 mM EDTA, and 0.1% SDS. The wash solution is replaced with fresh solution and incubated an additional 1.5 h at 60° C. Filters are blotted dry and exposed for autoradiography. If necessary, filters are washed for a third time at 65-68° C. and reexposed to film. Other conditions of low stringency which may be used are well known in the art (e.g., as employed for cross-species hybridizations).

Any technique for mutagenesis known in the art can be used to modify individual nucleotides in a DNA sequence, for purpose of making amino acid substitution(s) in the expressed peptide sequence, or for creating/deleting restriction sites to facilitate further manipulations. Such techniques include but are not limited to, chemical mutagenesis, in vitro site-directed mutagenesis (Hutchinson et al., 1978, J. Biol. Chem. 253: 6551), oligonucleotide-directed mutagenesis (Smith, 1985, Ann. Rev. Genet. 19: 423-463; Hill et al., 1987, Methods Enzymol. 155: 558-568), PCR-based overlap extension (Ho et al., 1989, Gene 77: 51-59), PCR-based megaprimer mutagenesis (Sarkar et al., 1990, Biotechniques 8: 404-407), etc. Modifications can be confirmed by double stranded dideoxynucleotide DNA sequencing.

In certain embodiments, a nucleic acid encoding a secretory form of a non-secreted HSP is used to practice the methods of the present invention. Such a nucleic acid can be constructed by deleting the coding sequence for the ER retention signal, KDEL. Optionally, the KDEL coding sequence is replaced with a molecular tag to facilitate the recognition and purification of the HSP, such as the Fc portion of murine IgG1. In another embodiment, a molecular tag can be added to naturally secreted HSPs or α2M. U.S. application Ser. No. 09/253,439 demonstrates that deletion of the ER retention signal of gp96 resulted in the secretion of gp96-Ig peptide-complexes from transfected tumor cells, and the fusion of the KDEL-deleted gp96 with murine IgG1 facilitated its detection by ELISA and FACS analysis and its purification by affinity chromatography with the aid of Protein A.

4.2.1.1. Expression Systems

Nucleotide sequences encoding an HSP or α2M molecule can be inserted into the expression vector for propagation and expression in recombinant cells. An expression construct, as used herein, refers to a nucleotide sequence encoding an HSP or α2M operably associated with one or more regulatory regions which allows expression of the HSP or α2M molecule in an appropriate host cell. “Operably-associated” refers to an association in which the regulatory regions and the HSP or α2M polypeptide sequence to be expressed are joined and positioned in such a way as to permit transcription, and ultimately, translation of the HSP or α2M sequence. A variety of expression vectors may be used for the expression of HSPs or α2M, including, but not limited to, plasmids, cosmids, phage, phagemids, or modified viruses. Examples include bacteriophages such as lambda derivatives, or plasmids such as pBR322 or pUC plasmid derivatives or the Bluescript vector (Stratagene). Typically, such expression vectors comprise a functional origin of replication for propagation of the vector in an appropriate host cell, one or more restriction endonuclease sites for insertion of the HSP or α2M gene sequence, and one or more selection markers.

For expression of HSPs or α2M in mammalian host cells, a variety of regulatory regions can be used, for example, the SV40 early and late promoters, the cytomegalovirus (CMV) immediate early promoter, and the Rous sarcoma virus long terminal repeat (RSV-LTR) promoter. Inducible promoters that may be useful in mammalian cells include but are not limited to those associated with the metallothionein II gene, mouse mammary tumor virus glucocorticoid responsive long terminal repeats (MMTV-LTR), the β-interferon gene, and the HSP70 gene (Williams et al., 1989, Cancer Res. 49: 2735-42; Taylor et al., 1990, Mol. Cell. Biol. 10: 165-75). The efficiency of expression of the HSP or α2M in a host cell may be enhanced by the inclusion of appropriate transcription enhancer elements in the expression vector, such as those found in SV40 virus, Hepatitis B virus, cytomegalovirus, immunoglobulin genes, metallothionein, α-actin (see Bittner et al., 1987, Methods in Enzymol. 153: 516-544; Gorman, 1990, Curr. Op. in Biotechnol. 1: 36-47).

The expression vector may also contain sequences that permit maintenance and replication of the vector in more than one type of host cell, or integration of the vector into the host chromosome. Such sequences may include but are not limited to replication origins, autonomously replicating sequences (ARS), centromere DNA, and telomere DNA. It may also be advantageous to use shuttle vectors that can be replicated and maintained in at least two types of host cells.

In addition, the expression vector may contain selectable or screenable marker genes for initially isolating or identifying host cells that contain DNA encoding an HSP or α2M. For long term, high yield production of HSPs or α2M, stable expression in mammalian cells is preferred. A number of selection systems may be used for mammalian cells, including, but not limited, to the Herpes simplex virus thymidine kinase (Wigler et al., 1977, Cell 11: 223), hypoxanthine-guanine phosphoribosyltransferase (Szybalski and Szybalski, 1962, Proc. Natl. Acad. Sci. U.S.A. 48: 2026), and adenine phosphoribosyltransferase (Lowy et al, 1980, Cell 22: 817) genes can be employed in tk⁻, hgprt⁻ or aprt⁻ cells, respectively. Also, antimetabolite resistance can be used as the basis of selection for dihydrofolate reductase (dhfr), which confers resistance to methotrexate (Wigler et al., 1980, Natl. Acad. Sci. U.S.A. 77: 3567; O'Hare et al., 1981, Proc. Natl. Acad. Sci. U.S.A. 78: 1527); gpt, which confers resistance to mycophenolic acid (Mulligan and Berg, 1981, Proc. Natl. Acad. Sci. U.S.A. 78: 2072); neomycin phosphotransferase (neo), which confers resistance to the aminoglycoside G-418 (Colberre-Garapin et al., 1981, J. Mol. Biol. 150: 1); and hygromycin phosphotransferase (hyg), which confers resistance to hygromycin (Santerre et al., 1984, Gene 30: 147). Other selectable markers, such as but not limited to histidinol and Zeocin™ can also be used.

The expression construct comprising an HSP- or α2M-coding sequence operably associated with regulatory regions can be directly introduced into appropriate host cells for expression and production of the HSP or α2M complexes of the invention without further cloning (see, for example, U.S. Pat. No. 5,580,859). The expression constructs may also contain DNA sequences that facilitate integration of the coding sequence into the genome of the host cell, e.g., via homologous recombination. In this instance, it is not necessary to employ an expression vector comprising a replication origin suitable for appropriate host cells in order to propagate and express the HSP or α2M molecule in the host cells.

Expression constructs containing cloned HSP or α2M coding sequences can be introduced into the mammalian host cell by a variety of techniques known in the art, including but not limited to calcium phosphate mediated transfection (Wigler et al., 1977, Cell 11: 223-232), liposome-mediated transfection (Schaefer-Ridder et al., 1982, Science 215: 166-168), electroporation (Wolff et al., 1987, Proc. Natl. Acad. Sci. 84: 3344), and microinjection (Cappechi, 1980, Cell 22: 479-488).

Any of the cloning and expression vectors described herein may be synthesized and assembled from known DNA sequences by techniques well known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

Alternatively, number of viral-based expression systems may also be utilized with mammalian cells for recombinant expression of HSPs or α2M. Vectors using DNA virus backbones have been derived from simian virus 40 (SV40) (Hamer et al., 1979, Cell 17: 725), adenovirus (Van Doren et al., 1984, Mol. Cell Biol. 4: 1653), adeno-associated virus (McLaughlin et al., 1988, J. Virol. 62: 1963), and bovine papillomas virus (Zinn et al., 1982, Proc. Natl. Acad. Sci. 79: 4897). In cases where an adenovirus is used as an expression vector, the donor DNA sequence may be ligated to an adenovirus transcription/translation control region, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing heterologous products in infected hosts (see, e.g., Logan and Shenk, 1984, Proc. Natl. Acad. Sci. U.S.A. 81: 3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene construct is transfected into cultured mammalian cells, for example, by the techniques of calcium phosphate coprecipitation or electroporation. For those host cells that do not manifest a transformed phenotype, selection of transformants is achieved by use of a dominant selectable marker, such as histidinol and G418 resistance. For example, BPV vectors such as pBCMGSNeo and pBCMGHis may be used to express HSPs or α2M (Karasuyama et al., Eur. J. Immunol. 18: 97-104; Ohe et al., Human Gene Therapy 6: 325-33) which may then be transfected into a diverse range of cell types for HSP or α2M expression.

Alternatively, the vaccinia 7.5K promoter may be used (see, e.g., Mackett et al., 1982, Proc. Natl. Acad. Sci. U.S.A. 79: 7415-7419; Mackett et al., 1984, J. Virol. 49: 857-864; Panicali et al., 1982, Proc. Natl. Acad. Sci. U.S.A. 79: 4927-4931) In cases where a human host cell is used, vectors based on the Epstein-Barr virus (EBV) origin (OriP) and EBV nuclear antigen 1 (EBNA-1; a trans-acting replication factor) may be used. Such vectors can be used with a broad range of human host cells, e.g., EBO-pCD (Spickofsky et al., 1990, DNA Prot. Eng. Tech. 2: 14-18), pDR2 and λDR2 (available from Clontech Laboratories).

Recombinant HSP or α2M expression can also be achieved by a retrovirus-based expression system. In contrast to transfection, retroviruses can efficiently infect and transfer genes to a wide range of cell types including, for example, primary hematopoietic cells. In retroviruses such as Moloney murine leukemia virus, most of the viral gene sequences can be removed and replaced with an HSP or α2M coding sequence, while the missing viral functions can be supplied in trans. The host range for infection by a retroviral vector can also be manipulated by the choice of envelope used for vector packaging.

For example, a retroviral vector can comprise a 5′ long terminal repeat (LTR), a 3′ LTR, a packaging signal, a bacterial origin of replication, and a selectable marker. The ND-associated antigenic peptide DNA is inserted into a position between the 5′ LTR and 3′ LTR, such that transcription from the 5′ LTR promoter transcribes the cloned DNA. The 5′ LTR comprises a promoter, including but not limited to an LTR promoter, an R region, a U5 region and a primer binding site, in that order. Nucleotide sequences of these LTR elements are well known in the art. A heterologous promoter as well as multiple drug selection markers may also be included in the expression vector to facilitate selection of infected cells (see McLauchlin et al., 1990, Prog. Nucleic Acid Res. and Molec. Biol. 38: 91-135; Morgenstern et al., 1990, Nucleic Acid Res. 18: 3587-3596; Choulika et al., 1996, J. Virol 70: 1792-1798; Boesen et al., 1994, Biotherapy 6: 291-302; Salmons and Gunzberg, 1993, Human Gene Therapy 4: 129-141; and Grossman and Wilson, 1993, Curr. Opin. in Genetics and Devel. 3: 110-114).

The recombinant cells may be cultured under standard conditions of temperature, incubation time, optical density, and media composition. Alternatively, cells may be cultured under conditions emulating the nutritional and physiological requirements of a cell in which the HSP is endogenously expressed. Modified culture conditions and media may be used to enhance production of HSP-peptide complexes. For example, recombinant cells may be grown under conditions that promote inducible HSP expression.

Alpha-2-macroglobulin and HSP polypeptides of the invention may be expressed as fusion proteins to facilitate recovery and purification from the cells in which they are expressed. For example, an HSP or α2M polypeptide may contain a signal sequence leader peptide to direct its translocation across the ER membrane for secretion into culture medium. Further, an HSP or α2M polypeptide may contain an affinity label, such as a affinity label, fused to any portion of the HSP or α2M polypeptide not involved in binding antigenic peptide, such as for example, the carboxyl terminal. The affinity label can be used to facilitate purification of the protein, by binding to an affinity partner molecule.

Various methods for production of such fusion proteins are well known in the art. The manipulations which result in their production can occur at the gene or protein level, preferably at the gene level. For example, the cloned coding region of an HSP or α2M polypeptide may be modified by any of numerous recombinant DNA methods known in the art (Sambrook et al., 1990, Molecular Cloning, A Laboratory Manual, 2d ed., Cold Spring Harbor Laboratory, Cold Spring Harbor, N.Y.; Ausubel et al., in Chapter 8 of Current Protocols in Molecular Biology, Greene Publishing Associates and Wiley Interscience, New York). It will be apparent from the following discussion that substitutions, deletions, insertions, or any combination thereof are introduced or combined to arrive at a final nucleotide sequence encoding an HSP or α2M polypeptide.

In various embodiments, fusion proteins comprising the HSP or α2M polypeptide may be made using recombinant DNA techniques. For example, a recombinant gene encoding an HSP or α2M polypeptide may be constructed by introducing an HSP or α2M gene fragment in the proper reading frame into a vector containing the sequence of an affinity label, such that the HSP or α2M polypeptide is expressed as a peptide-tagged fusion protein. Affinity labels, which may be recognized by specific binding partners, may be used for affinity purification of the HSP or α2M polypeptide.

In a preferred embodiment, the affinity label is fused at its amino terminal to the carboxyl terminal of HSP or α2M. The precise site at which the fusion is made in the carboxyl terminal is not critical. The optimal site can be determined by routine experimentation.

A variety of affinity labels known in the art may be used, such as, but not limited to, the immunoglobulin constant regions, polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other affinity labels may impart fluorescent properties to an HSP or α2M polypeptide, e.g., portions of green fluorescent protein and the like. Other possible affinity labels are short amino acid sequences to which monoclonal antibodies are available, such as but not limited to the following well known examples, the FLAG epitope, the myc epitope at amino acids 408-439, the influenza virus hemagglutinin (HA) epitope. Other affinity labels are recognized by specific binding partners and thus facilitate isolation by affinity binding to the binding partner which can be immobilized onto a solid support. Some affinity labels may afford the HSP or α2M polypeptide novel structural properties, such as the ability to form multimers. Dimerization of an HSP or α2M polypeptide with a bound peptide may increase avidity of interaction between the HSP or α2M polypeptide and its partner in the course of antigen presentation. These affinity labels are usually derived from proteins that normally exist as homopolymers. Affinity labels such as the extracellular domains of CD8 (Shiue et al., 1988, J. Exp. Med. 168:1993-2005), or CD28 (Lee et al., 1990, J. Immunol. 145:344-352), or portions of the immunoglobulin molecule containing sites for interchain disulfide bonds, could lead to the formation of multimers. As will be appreciated by those skilled in the art, many methods can be used to obtain the coding region of the above-mentioned affinity labels, including but not limited to, DNA cloning, DNA amplification, and synthetic methods. Some of the affinity labels and reagents for their detection and isolation are available commercially.

A preferred affinity label is a non-variable portion of the immunoglobulin molecule. Typically, such portions comprise at least a functionally operative CH2 and CH3 domain of the constant region of an immunoglobulin heavy chain. Fusions are also made using the carboxyl terminus of the Fc portion of a constant domain, or a region immediately amino-terminal to the CH1 of the heavy or light chain. Suitable immunoglobulin-based affinity label may be obtained from IgG-1, -2, -3, or -4 subtypes, IgA, IgE, IgD, or IgM, but preferably IgG1. Preferably, a human immunoglobulin is used when the HSP or α2M polypeptide is intended for in vivo use for humans. Many DNA encoding immunoglobulin light or heavy chain constant regions is known or readily available from cDNA libraries. See, for example, Adams et al., Biochemistry, 1980, 19:2711-2719; Gough et al., 1980, Biochemistry, 19:2702-2710; Dolby et al., 1980, Proc. Natl. Acad. Sci. U.S.A., 77:6027-6031; Rice et al., 1982, Proc. Natl. Acad. Sci. U.S.A., 79:7862-7865; Falkner et al., 1982, Nature, 298:286-288; and Morrison et al., 1984, Ann. Rev. Immunol, 2:239-256. Because many immunological reagents and labeling systems are available for the detection of immunoglobulins, the HSP or α2M polypeptide-Ig fusion protein can readily be detected and quantified by a variety of immunological techniques known in the art, such as the use of enzyme-linked immunosorbent assay (ELISA), immunoprecipitation, fluorescence activated cell sorting (FACS), etc. Similarly, if the affinity label is an epitope with readily available antibodies, such reagents can be used with the techniques mentioned above to detect, quantitate, and isolate the HSP or α2M polypeptide containing the affinity label. In many instances, there is no need to develop specific antibodies to the HSP or α2M polypeptide.

A particularly preferred embodiment is a fusion of an HSP or α2M polypeptide to the hinge, the CH2 and CH3 domains of human immunoglobulin G-1 (IgG1; see Bowen et al.,1996, J. Immunol. 156:442-49). This hinge region contains three cysteine residues which are normally involved in disulfide bonding with other cysteines in the Ig molecule. Since none of the cysteines are required for the peptide to function as a tag, one or more of these cysteine residues may optionally be substituted by another amino acid residue, such as for example, serine.

Various leader sequences known in the art can be used for the efficient secretion of HSP or α2M polypeptide from bacterial and mammalian cells (von Heijne, 1985, J. Mol. Biol. 184:99-105). Leader peptides are selected based on the intended host cell, and may include bacterial, yeast, viral, animal, and mammalian sequences. For example, the herpes virus glycoprotein D leader peptide is suitable for use in a variety of mammalian cells. A preferred leader peptide for use in mammalian cells can be obtained from the V-J2-C region of the mouse immunoglobulin kappa chain (Bernard et al., 1981, Proc. Natl. Acad. Sci. 78:5812-5816). Preferred leader sequences for targeting HSP or α2M polypeptide expression in bacterial cells include, but are not limited to, the leader sequences of the E.coli proteins OmpA (Hobom et al., 1995, Dev. Biol. Stand. 84:255-262), Pho A (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), and the Staphylococcus aureus protein A (Abrahmsen et al., 1986, Nucleic Acids Res. 14:7487-7500), and the B. subtilis endoglucanase (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

DNA sequences encoding a desired affinity label or leader peptide, which may be readily obtained from libraries, produced synthetically, or may be available from commercial suppliers, are suitable for the practice of this invention. Such methods are well known in the art.

4.3. Complexing Peptide Fragments to HSP and A2M

Described herein are methods for complexing in vitro the HSP or α2M with a population of peptides which have been generated by digestion of a protein preparation of antigenic cells. The complexing reaction can result in the formation of a covalent bond between an HSP and a peptide, or an α2M and a peptide. The complexing reaction can also result in the formation of a non-covalent association between an HSP and a peptide, or an α2M and a peptide.

In a method which produces non-covalent HSP-antigenic molecule complexes and α2M-antigenic molecule complexes, a complex is prepared according to the method described by Blachere et al., 1997 J. Exp. Med. 186(8):1315-22, which incorporated by reference herein in its entirety. Blachere teaches in vitro complexing of hsps to antigenic molecule. The protocol described in Blachere can be modified such that the hsp component is substituted by α2M. Binder et al. (2001, J. Immunol. 166:4968-72) demonstrates that the Blachere method yields complexes of α2M bound to antigenic molecules.

Prior to complexing, the HSPs can be pretreated with ATP or low pH to remove any peptides that may be non-covalently associated with the HSP of interest. When the ATP procedure is used, excess ATP is removed from the preparation by the addition of apyranase as described by Levy, et al., 1991, Cell 67:265-274. When the low pH procedure is used, the buffer is readjusted to neutral pH by the addition of pH modifying reagents. A preferred, exemplary protocol for the complexing of a population of peptides (average length between 7 to 20 amino acids) to an HSP or α2M in vitro is discussed below

The population of peptides (1 μg) and the pretreated HSP (9 μg) are admixed to give an approximately 5 peptides: 1 stress protein molar ratio. Then, the mixture is incubated for 15 minutes to 3 hours at 4° to 45° C. in a suitable binding buffer such as one containing 20 mM sodium phosphate, pH 7.2, 350 mM NaCl, 3 mM MgCl₂ and 1 mM phenyl methyl sulfonyl fluoride (PMSF). The preparations are centrifuged through a Centricon 10 assembly (Millipore) to remove any unbound peptide. The non-covalent association of the peptides with the stress proteins can be assayed by High Performance Liquid Chromatography (HPLC) or Mass Spectrometry (MS).

In an alternative embodiment of the invention, preferred for producing non-covalent complexes of HSP70 to peptide fragments: 5-10 micrograms of purified HSP70 is incubated with equimolar quantities of peptide fragments in 20 mM sodium phosphate buffer pH 7.5, 0.5M NaCl, 3 mM MgCl₂ and 1 mM ADP in a volume of 100 microliter at 37° C. for 1 hr. This incubation mixture is centrifuged one or more times if necessary, through a Centricon 10 assembly (Millipore) to remove any unbound peptide.

In an alternative embodiment of the invention, preferred for producing complexes of gp96 or HSP90 to peptide fragments, 5-10 micrograms of purified gp96 or HSP90 is incubated with equimolar or excess quantities of the peptide fragments in a suitable buffer such as one containing 20 mM sodium phosphate buffer pH 7.5, 0.5M NaCl, 3 mM MgCl2 at 60-65° C. for 5-20 min. This incubation mixture is allowed to cool to room temperature and centrifuged one or more times if necessary, through a Centricon 10 assembly (Millipore) to remove any unbound peptide.

Following complexing, an immunogenic HSP-peptide complex or α2M-peptide complex can optionally be assayed using for example the mixed lymphocyte target cell assay (MLTC) described below. Once HSP-peptide complexes have been isolated and diluted, they can be optionally characterized further in animal models using the preferred administration protocols and excipients discussed below.

As an alternative to making non-covalent complexes of HSPs and peptides, a population of peptides can be covalently attached to HSPs. Covalently linked complexes are the complexes of choice when a B cell response is desired.

In one embodiment, HSPs are covalently coupled to peptide fragments by chemical crosslinking. Chemical crosslinking methods are well known in the art. For example, in a preferred embodiment, glutaraldehyde crosslinking may be used. Glutaradehyde crosslinking has been used for formation of covalent complexes of peptides and HSPs (see Barrios et al., 1992, Eur. J. Immunol. 22: 1365-1372). Preferably, 1-2 mg of HSP-peptide complex is crosslinked in the presence of 0.002% glutaraldehyde for 2 hours. Glutaraldehyde is removed by dialysis against phosphate buffered saline (PBS) overnight (Lussow et al., 1991, Eur. J. Immunol. 21: 2297-2302). Alternatively, an HSP and a population of peptides can be crosslinked by ultraviolet (UV) crosslinking under conditions known in the art. In another embodiment of the invention, a population of peptides can be complexed to α2M by incubating the peptide fragments with α2M at a 50:1 molar ratio and incubated at 50° C. for 10 minutes followed by a 30 minute incubation at 25° C. Free (uncomplexed) peptides are then removed by size exclusion filters. Protein-peptide complexes are preferably measured by a scintillation counter to make sure that on a per molar basis, each protein is observed to bind equivalent amounts of peptide (approximately 0.1% of the starting amount of the peptide). For details, see Binder, 2001, J. Immunol. 166(8):4968-72, which is incorporated herein by reference in its entirety.

Alternatively, a population of antigenic peptides can be complexed to α2M covalently by methods as described in PCT publications WO 94/14976 and WO 99/50303 for complexing a peptide to α2M, which are incorporated herein by reference in their entirety. Covalent linking of a population of antigenic peptides to α2M can be performed using a bifunctional crosslinking agent. Such crosslinking agents and methods of their use are also well known in the art.

4.4. HSP/α2M Vaccine Compositions that Can Be Used

The HSP/α2M vaccine compositions that can be used with the HSP or α2M preparations of the invention can include but are not limited to HSP/α2M-peptide complexes complexed to tumor antigens, e.g., containing tumor specific or tumor-associated antigens which have been developed for the treatment or prevention of various types of cancers, or complexes isolated from cancerous tissues or cancer cell lines. Non-limiting examples of tumor antigens that can be used in a vaccine composition may include KS 1/4 pan-carcinoma antigen (Perez and Walker, 1990, J. Immunol. 142:3662-3667; Bumal, 1988, Hybridoma 7(4):407-415); ovarian carcinoma antigen (CA125) (Yu, et al., 1991, Cancer Res. 51(2):468-475); prostatic acid phosphate (Tailer, et al., 1990, Nucl. Acids Res. 18(16):4928); prostate specific antigen (Henttu and Vihko, 1989, Biochem. Biophys. Res. Comm. 160(2):903-910; Israeli, et al., 1993, Cancer Res. 53:227-230); melanoma-associated antigen p97 (Estin, et al., 1989, J. Natl. Cancer Inst. 81(6):445-446); melanoma antigen gp75 (Vijayasardahl, et al., 1990, J. Exp. Med. 171(4):1375-1380); high molecular weight melanoma antigen (Natali, et al., 1987, Cancer 59:55-63), the MAGE family of antigens (Hu et al., 1996, Cancer Res. 56:2479-2483; Marchand et al., 1995, Int. J. Cancer 63:883-885) and prostate specific membrane antigen.

The HSP/α2M vaccine compositions useful for the treatment or prevention of infectious diseases can comprise HSP or α2M complexed to a known antigen associated with an agent of a particular infectious disease, or complexed to a molecule displaying the antigencity of such an antigen, or can be isolated from infected cells.

The HSP/α2M vaccine compositions of the invention may be autologous HSP-peptide complexes and α2M peptide-complexes, or the HSP/α2M component or the peptide component of the complexes may be autologous; i.e., isolated from the patient to which they are administered. Alternatively, the HSP/α2M vaccine compositions or components thereof may be allogeneic.

Many methods may be used to introduce the vaccine; these include but are not limited to oral, intradermal, intramuscular, intraperitoneal, intravenous, subcutaneous, intranasal routes, and via scarification (scratching through the top layers of skin, e.g., using a bifurcated needle).

The patient to which the vaccine is administered is preferably a mammal, most preferably a human, but can also be a non-human animal including but not limited to primates, cows, horses, sheep, pigs, fowl (e.g., chickens), goats, cats, dogs, hamsters, mice and rats.

The HSP/α2M vaccine composition may comprise adjuvants, or may be administered together with one or more adjuvants. Adjuvants that can be used include but are not limited to mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum hydroxide, aluminum phosphate gel, Freund's Complete Adjuvant, Freund's Incomplete Adjuvant, squalene or squalane oil-in-water adjuvant formulations, biodegradable and biocompatible polyesters, polymerized liposomes, triterpenoid glycosides or saponins (e.g., QuilA and QS-21, also sold under the trademark STIMULON, ISCOPREP), N-acetyl-muramyl-L-threonyl-D-isoglutamine (Threonyl-MDP, sold under the trademark TERMURTIDE), LPS, monophosphoryl Lipid A (3D-MLAsold under the trademark MPL).

4.5. Kits, Dosage Regimens, Administration and Formulations

Kits are also provided for carrying out the vaccination methods of the present invention. In a specific embodiment, a kit comprises a first container containing a heat shock protein preparation or an α2M preparation in an amount effective to increase or prolong an immune response elicited by an HSP/α2M vaccine composition against a component of the HSP/α2M vaccine composition against which an immune response is desired; and a second container containing the HSP/α2M vaccine composition in an amount that, when administered before, concurrently with, or after the administration of the heat shock protein preparation or the α2M preparation in the first container, is effective to induce an immune response against the component. In an alternate embodiment, the kit comprises a container containing both the HSP preparation and the HSP/α2M vaccine composition, wherein the HSP preparation and the HSP/α2M vaccine composition are not present in admixture. In another alternate embodiment, the kit comprises a container containing both the α2M preparation and the HSP/α2M vaccine composition, wherein the α2M preparation and the HSP/α2M vaccine composition are not present in admixture.

Kits of the invention are provided that comprise in a container an HSP/α2M vaccine composition in an amount effective to treat or prevent a disease or disorder; and in another container either a heat shock protein preparation or an α2M preparation in an amount effective to increase or prolong an immune response elicited by the vaccine. In an embodiment, the amount of HSP/α2M vaccine composition present in the container is insufficient for inducing an immune response in a subject if administered independent of the heat shock protein preparation or of the α2M preparation in the other container. The kit may optionally be accompanied by instructions.

The dosage of HSP preparation or α2M preparation to be administered depends to a large extent on the condition and size of the subject being treated as well as the amount of HSP/α2M vaccine composition administered, the frequency of treatment and the route of administration. Regimens for continuing therapy, including site, dose and frequency may be guided by the initial response and clinical judgment.

Depending on the route of administration and the type of HSPs in the HSP preparation, the amount of HSP in the HSP preparation can range, for example, from 0.1 to 1000 μg per administration. The preferred amounts of gp96 or hsp70 are in the range of 10 to 600 μg per administration and 0.1 to 10 μg if the HSP preparation is administered intradermally. For hsp 90, the preferred amounts are about 50 to 1000 μg per administration, and about 5 to 50 μg for intradermal administration. The amount of α2M administered can range from 2 to 1000 μg, preferably 20 to 500 μg, most preferably about 25 to 250 μg, given once weekly for about 4-6 weeks, intradermally with the site of administration varied sequentially.

In one preferred embodiment, the HSP preparation or the α2M preparation is administered concurrently with the administration of a vaccine. Concurrent administration of an HSP preparation or α2M preparation and a vaccine means that the HSP or α2M preparation is given at reasonably the same time as the vaccine. This method provides that the two administrations are performed within a time frame of less than one minute to about five minutes, or up to about sixty minutes from each other, for example, at the same doctor's visit.

Because of the administration of the HSP preparation or the α2M preparation, lesser amount of vaccine is required to elicit an immune respone in a subject. In specific embodiments, a reduction of about 10%, 20%, 30%, 40% and 50% of the amount of HSP/α2M vaccine composition can be achieved. Even sub-immunogenic amounts of the HSP/α2M vaccine composition can be used provided that an appropriate amount of the HSP preparation or α2M preparation is used in conjunction. The amount of HSP/α2M vaccine composition to be used with an HSP preparation or α2M preparation, including amounts in the sub-immunogenic range, can be determined by dose-response experiments conducted in animal models by methods well known in the art.

Solubility and the site of the vaccination are factors which should be considered when choosing the route of administration of the HSP or α2M preparation of the invention. The mode of administration can be varied, including, but not limited to, e.g., subcutaneously, intravenously, intraperitoneally, intramuscularly, intradermally or mucosally. Mucosal routes can further take the form of oral, rectal and nasal administration. With the above factors taken into account, it is preferable to administer the HSP or the α2M to a site that is the same or proximal to the site of vaccination.

In an embodiment of the invention, HSPs or α2M may be administered using any desired route of administration. Advantages of intradermal administration include use of lower doses and rapid absorption, respectively. Advantages of subcutaneous or intramuscular administration include suitability for some insoluble suspensions and oily suspensions, respectively. Mucosal routes of administration include, but are not limited to, oral, rectal and nasal administration. Preparations for mucosal administrations are suitable in various formulations as described below.

In a preferred embodiment, the invention provides for a method of introducing an HSP preparation including, but not limited to, hsp70, hsp90 and gp96 alone or in combination with each other into a subject concurrently with the administration of a vaccine at the same site or at a site in close proximity. In another preferred embodiment, the invention provides for a method of introducing an α2M preparation concurrently with the administration of a vaccine at the same site or at a site in close proximity. Preferably the HSP preparation or the α2M preparation are not administered with the HSP/α2M vaccine composition in admixture.

If the HSP or α2M preparation is water-soluble, then it may be formulated in an appropriate buffer, for example, phosphate buffered saline or other physiologically compatible solutions, preferably sterile. Alternatively, if the resulting complex has poor solubility in aqueous solvents, then it may be formulated with a non-ionic surfactant such as Tween, or polyethylene glycol. Thus, the compounds and their physiologically acceptable solvates may be formulated for administration by inhalation or insufflation (either through the mouth or the nose) or oral, buccal, parenteral, or rectal administration or, in the case of tumors, directly injected into a solid tumor.

For oral administration, the pharmaceutical preparation may be in liquid form, for example, solutions, syrups or suspensions, or may be presented as a drug product for reconstitution with water or other suitable vehicle before use. Such a liquid preparation may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-phydroxybenzoates or sorbic acid). The pharmaceutical preparation may take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

The HSP preparation for oral administration may be suitably formulated to give controlled release of the active compound.

For buccal administration, the preparation may take the form of tablets or lozenges formulated in conventional manner.

The preparation may be formulated for parenteral administration by injection, e.g., by bolus injection or continuous infusion. Formulations for injection may be presented in unit dosage form, e.g., in ampoules or in multi-dose containers, with an added preservative. The preparation may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The preparation may also be formulated in a rectal preparation such as a suppository or retention enema, e.g., containing conventional suppository bases such as cocoa butter or other glycerides.

In addition to the formulations described previously, the preparation may also be formulated as a depot preparation. Such long acting formulations may be administered by implantation (for example, subcutaneously or intramuscularly) or by intramuscular injection. Thus, for example, the preparation may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

For administration by inhalation, the preparation for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The preparation may, if desired, be presented in a pack or dispenser device which may contain one or more unit dosage forms containing the HSP preparation or the α2M preparation. The pack may for example comprise metal or plastic foil, such as a blister pack. The pack or dispenser device may be accompanied by instructions for administration.

4.6. Determination of Immunogenicity of Vaccines After HSP or A2M Treatment

In an optional procedure, the production of or increase in immunogenicity of a vaccine that is used with the HSP or α2M preparation of the invention can be assessed using various methods well known in the art.

In one method, the immunogenicity of the vaccine and HSP preparation or α2M preparation is determined by measuring antibodies produced in response, by an antibody assay, such as an enzyme-linked immunosorbent assay (ELISA) assay. Methods for such assays are well known in the art (see, e.g., Section 2.1 of Current Protocols in Immunology, Coligan et al. (eds.), John Wiley and Sons, Inc. 1997). For example, microtitre plates (96-well Immuno Plate II, Nunc) are coated with 50 μl/well of a 0.75 μg/ml extract or lysate of a cancer cell or infected cell in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or CSF from a vaccinated animal (such as a model mouse or a human patient with or without administration of an HSP preparation) is applied at 20° C. for 1 hour, and the plates are washed 3 times with PBS-T. The antigen antibody activity is then measured colorimetrically after incubating at 20° C. for 1 hour with 50 μl/well of sheep anti-mouse or anti-human immunoglobulin, as appropriate, conjugated with horseradish peroxidase diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a photometer at 492 nm (ref. 620 nm), using standard techniques.

In another method, the “tetramer staining” assay (Altman et al., 1996, Science 274: 94-96) may be used to identify antigen-specific T cells. For example, in one embodiment, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of T cells obtained from a patient treated with a vaccine and the HSP preparation. Biotin is then used to stain T cells which express the tumor-specific antigen of interest.

Furthermore, using the mixed lymphocyte target culture assay, the cytotoxicity of T cells can be tested in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are pre-labeled by incubating 1×10⁶ target cells in culture medium containing 500 μCi of ⁵¹Cr per ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelleted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm. In order to block the MHC class I cascade a concentrated hybridoma supernatant derived from K-44 hybridoma cells (an anti-MHC class I hybridoma) is added to the test samples to a final concentration of 12.5%.

Alternatively, the ELISPOT assay can be used to measure cytokine release in vitro by cytotoxic T cells after stimulation with vaccine and HSP preparation or α2M preparation. Cytokine release is detected by antibodies which are specific for a particular cytokine, such as interleukin-2, tumor necrosis factor α or interferon-γ (for example, see Scheibenbogen et al., 1997, Int. J. Cancer, 71:932-936). The assay is carried out in a microtitre plate which has been pre-coated with an antibody specific for a cytokine of interest which captures the cytokine secreted by T cells. After incubation of T cells for 24-48 hours in the coated wells, the cytotoxic T cells are removed and replaced with a second labeled antibody that recognizes a different epitope on the cytokine. After extensive washing to remove unbound antibody, an enzyme substrate which produces a colored reaction product is added to the plate. The number of cytokine-producing cells is counted under a microscope. This method has the advantages of short assay time, and sensitivity without the need of a large number of cytotoxic T cells.

4.7. Treatment and Prevention of Infectious Diseases

Infectious diseases that can be treated or prevented by use of an HSP/α2M vaccine composition in conjunction with the methods of the present invention are caused by infectious agents including, but not limited to, viruses, bacteria, fungi protozoa and parasites.

Viral diseases that can be treated or prevented by use of an HSP/α2M vaccine composition in conjunction with the methods of the present invention include, but are not limited to, those caused by hepatitis A virus, hepatitis B virus, hepatitis C virus, influenza, varicella, adenovirus, herpes simplex I virus, herpes simplex II virus, rinderpest, rhinovirus, echovirus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I), and human immunodeficiency virus type II (HIV-II).

Bacterial diseases that can be treated or prevented by use of an HSP/α2M vaccine composition in conjunction with the methods of the present invention are caused by bacteria including, but not limited to, mycobacteria rickettsia, mycoplasma, neisseria and legionella.

Protozoal diseases that can be treated or prevented by use of an HSP/α2M vaccine composition in conjunction with the methods of the present invention are caused by protozoa including, but not limited to, leishmania, kokzidioa, and trypanosoma.

Parasitic diseases that can be treated or prevented by use of an HSP/α2M vaccine composition in conjunction with the methods of the present invention are caused by parasites including, but not limited to, chlamydia and rickettsia.

4.8. Treatment of Cancer

Cancers that can be treated by use of an HSP/α2M vaccine composition in conjunction with the methods of the present invention include, but are not limited to the following types of cancer: human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease.

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for inducing an immune response in a subject comprising the steps of:
(a) administering to the subject an HSP/α2M vaccine composition comprising an HSP or α2M complexed to a component against which an immune response is desired to be induced; and
(b) administering to the subject a heat shock protein preparation, wherein the heat shock protein preparation does not display the immunogenicity of the component, and wherein the heat shock protein preparation and HSP/α2M vaccine composition are not present in admixture;
such that an immune response to the component is produced in the subject.

2. A method of inducing an immune response by an HSP/α2M vaccine composition in a subject comprising the steps of:
(a) administering to the subject a heat shock protein preparation; and
(b) administering to the subject an HSP/α2M vaccine composition comprising an HSP or α2M complexed to a component against which an immune response is desired to be induced, the HSP/α2M vaccine composition being in an amount that is sub-immunogenic for the component in the absence of step (a),
such that an immune response to the component is induced in the subject, and wherein the heat shock protein preparation does not display the immunogenicity of the component.

3. The method of claim 2, wherein the HSP/α2M vaccine composition and the heat shock protein preparation are not present in admixture.

4. A method of treating or preventing an infectious disease in a subject comprising the steps of:
(a) administering to the subject an HSP/α2M vaccine composition comprising an HSP or α2M complexed to a component that displays the antigenicity of an antigen of an infectious agent that causes the infectious disease; and
(b) administering to the subject an amount of a heat shock protein preparation effective in combination with step (a) to induce or increase an immune response to the component in the subject, wherein the heat shock protein preparation does not display the immunogenicity of the component, and wherein the HSP preparation and HSP/α2M vaccine composition are not present in admixture.

5. A method of treating or preventing a cancer in a subject comprising the steps of:
(a) administering to the subject an HSP/α2M vaccine composition comprising an HSP or α2M complexed to a component that displays the antigenicity of a tumor specific or tumor associated antigen of a cancer cell; and
(b) administering to the subject an amount of a heat shock protein preparation effective to induce or increase an immune response in the subject to the component, wherein the heat shock protein preparation does not display the immunogenicity of the component, and wherein the HSP preparation and HSP/α2M vaccine composition are not present in admixture.

6. The method of claim 1, wherein the immune response to the component produced in the subject is increased relative to the immune response to the component in the subject in the absence of step (b).

7. The method of claim 1 wherein the heat shock protein preparation comprises a heat shock protein selected from the group consisting of hsp70, hsp90, gp96, hsp110, grp170, calreticulin, and a combination thereof.

8. The method of claim 2 wherein the heat shock protein preparation comprises a heat shock protein selected group consisting of hsp70, hsp90, gp96, hsp110, grp170, calreticulin, and a combination thereof.

9. The method of claim 4 wherein the heat shock protein preparation comprises a heat shock protein selected from the group consisting of hsp70, hsp90, gp96, hsp110, grp170, calreticulin, and a combination thereof.

10. The method of claim 5 wherein the heat shock protein preparation comprises a heat shock protein selected from the group consisting of hsp70, hsp90, gp96, hsp110, grp170, calreticulin, and a combination thereof.

11. The method of claim 1 wherein the heat shock protein preparation comprises heat shock protein-peptide complexes.

12. The method of claim 2 wherein the heat shock protein preparation comprises heat shock protein-peptide complexes.

13. The method of claim 4 wherein the heat shock protein preparation comprises heat shock protein-peptide complexes.

14. The method of claim 5 wherein the heat shock protein preparation comprises heat shock protein-peptide complexes.

15. The method of claim 1 wherein the heat shock protein preparation comprises purified heat shock proteins.

16. The method of claim 2 wherein the heat shock protein preparation comprises purified heat shock proteins.

17. The method of claim 4 wherein the heat shock protein preparation comprises purified heat shock proteins.

18. The method of claim 5 wherein the heat shock protein preparation comprises purified heat shock proteins.

19. The method of claim 1 wherein the subject is human and the heat shock protein preparation comprises mammalian heat shock proteins.

20. The method of claim 2 wherein the subject is human and the heat shock protein preparation comprises mammalian heat shock proteins.

21. The method of claim 4 wherein the subject is human and the heat shock protein preparation comprises mammalian heat shock proteins.

22. The method of claim 5 wherein the subject is human and the heat shock protein preparation comprises mammalian heat shock proteins.

23. The method of claim 1, 2, 4, or 5 wherein the heat shock protein preparation is administered before the administration of the HSP/α2M vaccine composition.

24. The method of claim 1, 2, 4, or 5 wherein the heat shock protein preparation is administered concurrently with the administration of the HSP/α2M vaccine composition, and the heat shock protein preparation and the HSP/α2M vaccine composition are not present in admixture.

25. The method of claim 1, 2, 4, or 5 wherein the heat shock protein preparation is administered after the administration of the HSP/α2M vaccine composition.

26. The method of claim 7, 8, 9, or 10 wherein the heat shock protein preparation is administered before the administration of the HSP/α2M vaccine composition.

27. The method of claim 7, 8, 9, or 10 wherein the heat shock protein preparation is administered concurrently with the administration of the HSP/α2M vaccine composition, and the heat shock protein preparation and the HSP/α2M vaccine composition are not administered in admixture.

28. The method of claim 7, 8, 9, or 10 wherein the heat shock protein preparation is administered after the administration of the HSP/α2M vaccine composition.

29. The method of claim 11, 12, 13 or 14 wherein the heat shock protein preparation is administered before the administration of the HSP/α2M vaccine composition.

30. The method of claim 11, 12, 13 or 14 wherein the heat shock protein preparation is administered concurrently with the administration of the HSP/α2M vaccine composition, and the heat shock protein preparation and the HSP/α2M vaccine composition are not administered in admixture.

31. The method of claim 11, 12, 13 or 14 wherein the heat shock protein preparation is administered after the administration of the HSP/α2M vaccine composition.

32. The method of claim 15, 16, 17 or 18 wherein the heat shock protein preparation is administered before the administration of the HSP/α2M vaccine composition.

33. The method of claim 15, 16, 17 or 18 wherein the heat shock protein preparation is administered concurrently with the administration of the HSP/α2M vaccine composition, and the heat shock protein preparation and the HSP/α2M vaccine composition are not administered in admixture.

34. The method of claim 15, 16, 17 or 18 wherein the heat shock protein preparation is administered after the administration of the HSP/α2M vaccine composition.

35. The method of claim 4 wherein the infectious disease is selected from the group consisting of hepatitis A virus, hepatitis B virus, hepatitis C virus, influenza, varicella, adenovirus, herpes simplex I virus, herpes simplex II virus, rinderpest, rhinovirus, ECHO virus, rotavirus, respiratory syncytial virus, papilloma virus, papova virus, cytomegalovirus, echinovirus, arbovirus, hantavirus, coxsackie virus, mumps virus, measles virus, rubella virus, polio virus, human immunodeficiency virus type I (HIV-I), human immunodeficiency virus type II (HIV-II), mycobacteria, rickettsia, mycoplasma, neisseria, legionella, leishmania, kokzidioa, trypanosoma and chlamydia.

36. The method of claim 5 wherein the cancer is selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma, leukemia, acute lymphocytic leukemia, acute myelocytic leukemia, myeloblastic leukemia, promyelocytic leukemia, myelomonocytic leukemia, monocytic leukemia and erythroleukemia, chronic leukemia, chronic myelocytic leukemia, chronic granulocytic leukemia, chronic lymphocytic leukemia, polycythemia vera, lymphoma, Hodgkin's disease lymphoma, non-Hodgkin's disease lymphoma, multiple myeloma, Waldenström's macroglobulinemia, and heavy chain disease.

37. The method of claim 4, wherein the method is for preventing an infectious disease.

38. The method of claim 5, wherein the method is for treating a cancer.

39. The method of claim 5, wherein the method is for preventing a cancer.

40. A kit comprising:
(a) a first container containing a heat shock protein preparation or an α2M preparation, in an amount effective to increase an immune response elicited by an HSP/α2M vaccine composition against a component of the HSP/α2M vaccine composition against which an immune response is desired; and
(b) a second container containing the HSP/α2M vaccine composition in an amount that, when administered before, concurrently with, or after the administration of the heat shock protein preparation of (a), is effective to induce an immune response against the component.

41. The kit of claim 40 wherein the first container contains a heat shock protein preparation comprising a heat shock protein selected from the group consisting of hsp70, hsp90, gp96, hsp110, grp170, calreticulin, and a combination thereof.

42. The kit of claim 40 wherein the first container contains a heat shock protein preparation comprising heat shock protein-peptide complexes.

43. The kit of claim 40 wherein the first container contains a heat shock protein preparation comprising purified heat shock proteins.

44. The kit of claim 40 wherein the first container contains a heat shock protein preparation comprising heat shock protein-peptide complexes and purified heat shock proteins.

45. The kit of claim 40 wherein the first container contains a heat shock protein preparation comprising mammalian heat shock proteins.

46. The kit of claim 40 wherein the amount of HSP/α2M vaccine composition in the second container is insufficient for inducing an immune response in a subject in the absence of administering the heat shock protein preparation or α2M preparation in the first container.

47. A method for inducing an immune response in a subject comprising the steps of:
(a) administering to the subject an HSP/α2M vaccine composition comprising an HSP or α2M complexed to a component against which an immune response is desired to be induced; and
(b) administering to the subject an α2M preparation, wherein the α2M preparation does not display the immunogenicity of the component, and wherein the α2M preparation and the HSP/α2M vaccine composition are not present in admixture;
such that an immune response to the component is produced in the subject.

48. A method of inducing an immune response by an HSP/α2M vaccine composition in a subject comprising the steps of:
(a) administering to the subject an α2M preparation; and
(b) administering to the subject an HSP/α2M vaccine composition comprising an HSP or α2M complexed to a component against which an immune response is desired to be induced, the HSP/α2M vaccine composition being in an amount that is sub-immunogenic for the component in the absence of step (a),
such that an immune response to the component is induced in the subject, and wherein the α2M preparation does not display the immunogenicity of the component.

49. A method of treating or preventing an infectious disease in a subject comprising the steps of:
(a) administering to the subject an HSP/α2M vaccine composition comprising an HSP or α2M complexed to a component that displays the antigenicity an antigen of an infectious agent that causes the infectious disease; and
(b) administering to the subject an amount of an α2M preparation effective in combination with step (a) to induce or increase an immune response to the component in the subject, wherein the α2M preparation does not display the immunogenicity of the component, and wherein the HSP/α2M vaccine composition and α2M preparation are not present in admixture.

50. A method of treating or preventing a cancer in a subject comprising the steps of:
(a) administering to the subject an HSP/α2M vaccine composition comprising an HSP or α2M complexed to a component that displays the antigenicity of a tumor specific or tumor associated antigen of a cancer cell; and
(b) administering to the subject an amount of an α2M preparation effective to induce or increase an immune response in the subject to the component, wherein the α2M preparation does not display the immunogenicity of the component, and wherein the HSP/α2M vaccine composition and α2M preparation are not present in admixture.

51. The method of claim 47, wherein the immune response to the component produced in the subject is increased relative to the immune response to the component in the subject in the absence of step (b).

52. The method of claim 48, wherein the HSP/α2M vaccine composition and the α2M preparation are not present in admixture.

53. The method of claim 47, 48, 49, or 50 wherein the α2M preparation comprises α2M-peptide complexes.

54. The method of claim 47, 48, 49, or 50 wherein the α2M preparation comprises purified α2M.

55. The method of claim 47, 48, 49, or 50 wherein the subject is human and the α2M preparation comprises mammalian α2M.
